(19) United States
(12) Patent Application Publication
(10) Pub. No.: US 2024/0416018 A1
(43) Pub. Date: Dec. 19, 2024

(54) BLOOD DIALYZING APPARATUS AND METHOD

(71) Applicant: Exorenal Inc, Baltimore (MD)

(72) Inventor: Jake Kyungsoo Lee, Ellicott City, MD (US)

(21) Appl. No.: 18/759,894

(22) Filed: Jun. 30, 2024

Related U.S. Application Data
(63) Continuation-in-part of application No. 17/082,016, filed on Oct. 28, 2020.
(63) Continuation-in-part of application No. 17/893,149, filed on Aug. 22, 2022.
(60) Provisional application No. 63/524,627, filed on Jul. 1, 2023.
(60) Provisional application No. 63/540,107, filed on Sep. 24, 2023.
(60) Provisional application No. 63/611,118, filed on Dec. 16, 2023.
(60) Provisional application No. 63/626,326, filed on Jan. 29, 2024.

(51) Int. Cl. A61M 1/16; A61M 1/26; A61M 39/24

(52) U.S. Cl. CPC A61M 1/1621; A61M 1/267; A61M 39/24

(57) ABSTRACT

Provided is a blood dialyzing apparatus having multiple fluid chambers each having an internal space to accommodate dialysate, a chamber pressurizing member compressing or expanding the internal spaces of the chambers, and a chamber pressurizing member driver driving the chamber pressurizing member. The chambers are each connected with an inflow tube through which a fluid is provided to the chamber and an outflow tube through which a fluid of the chamber is discharged therefrom. Multiple flow control valves are also provided to regulate flow passages through the tubes.

### CROSS-REFERENCE TO RELATED APPLICATIONS

This application is a continuation-in-part of U.S. patent application Ser. No. 17/082,016 filed on Oct. 28, 2020, which is a continuation-in-part of U.S. patent application Ser. No. 16/573,934 filed on Sep. 17, 2019, now patented with Ser. No. 11,185,620, and 16703,757 filed on Dec. 4, 2019,
this application is a continuation-in-part of U.S. patent application Ser. No. 17/893,149 filed on Aug. 23, 2022, which is also a continuation-in-part of U.S. patent application Ser. No. 16/703,757 filed on Dec. 4, 2019 and Ser. No. 17/082,016 filed on Oct. 28, 2020, and
this application also claims the benefit of priority to U.S. Provisional Patent application Ser. Nos. 63/524,627 filed on Jul. 1, 2023, 63/540,107 filed on Sep. 24, 2023, 63/611,118 filed on Dec. 16, 2023, and 63/626,326 filed on Jan. 29, 2024, the entire contents of which are incorporated by reference herein.

### TECHNICAL FIELD

The present invention relates to blood dialyzing apparatus and method, in which a plurality of dialysate chambers is compressed and expanded to allow dialysate to flow through a blood dialyzing filter, thereby making the blood dialyzing apparatus simplified and light-weighted, providing easy operation, reducing cost for the dialyzing treatment, and eventually enabling the dialyzing treatment to be conducted at home.

### BACKGROUND

When there is a kidney dysfunction, water and waste products that have to be discharged out of body accumulate in blood and imbalance of electrolytes in the body occurs. Most commonly performed to improve such a kidney failure symptom, is hemodialysis which is to circulate blood out of body and rid the blood of the accumulated uremic toxin and excess water by a semi-permeable dialysis membrane. Hemodialysis is a method of seeking an electrolyte balance and ridding the body fluid of uremic toxin and excess water, taking advantages of diffusion applied due to the concentration difference and filtration applied due to the pressure difference between blood and dialysate.

Hemodialysis is the example of the blood dialyzing treatment in which blood of a patient is circulated extracorporeally to remove toxic substances from or supply beneficial ingredients to the blood. The blood dialyzing treatment is frequently combined with a blood dialyzing filter in which mass transfer between blood (i.e., a physiologic body fluid) and dialysate (i.e., a purified sterile solution).

Most commonly used of blood dialyzing filter is the type that is a cylinder-shaped container charged with a bundle of hollow fiber membranes and port-processed at both ends thereof by use of a synthetic resin like polyurethane. It is because the hollow fiber blood dialyzing filter has excellent mass-transfer efficiency resulting from large effective surface area between blood and dialysate compared to the small size as a whole.

Conventional blood dialyzing devices require a balancing unit connected to the multiple dialysate tubes, two or more dialysate pumps to transfer dialysate, and a blood pump to transfer blood of a patient. It is also indispensable to disinfect the balancing unit, the dialysate pumps, and the dialysate flowing tubes on a regular basis, rendering the conventional blood dialyzing unit complex in the structure and complicated to use.

U.S. Pat. No. 4,054,522 discloses a degassing apparatus employing multiple fluid chambers, through which dialysate sequentially flows and therefore, functioning as a dialysate pump. The fluid chambers are pressurized or expanded by the piston pushing a rolling diaphragm. In addition, the diaphragm pump having four diaphragm chambers is used to transfer blood, according to WO 8,601,115, in which the diaphragm chambers are operated by a pneumatic driver. However, despite using multiple fluid chambers, they are neither able to achieve the balancing accuracy nor regulate the net volume removal. Additional separate ultrafiltration pumps or balancing chambers are required.

The prior disclosures only focus on transferring the fluid, such as either blood or dialysate, and therefore, they are limited to using the diaphragm type pumps. Unlike the disclosure, the four diaphragm chambers are not able to ensure blood flow rates equal upstream and downstream of the dialyzer due to flow resistances through the arterial blood circuit.

In order to solve the aforementioned problems, a novel blood dialyzing apparatus is provided, in which multiple blood chambers are compressed and expanded to transfer blood. The multiple chambers ensure blood flow rates upstream and downstream of the blood dialyzing filter to be regulated independently. Neither separate blood pump nor a separate ultrafiltration pump, nor a separate balancing chamber are required. Therefore, the entire system can be sufficiently miniaturized and light-weighted, and easy to be installed while reducing the cost for blood dialyzing treatment. The blood dialyzing apparatus will eventually be an optimal alternative for the blood dialyzing treatment in a place out of hospitals.

### SUMMARY

The blood dialyzing apparatus according to an embodiment of the present invention is configured to include a blood dialyzing filter, a blood flowing unit through which blood is supplied to the blood dialyzing filter and returned to the patient, and a dialysate flowing unit where dialysate is prepared and transferred through a blood dialyzing filter. The dialysate flowing unit further includes a flow control unit that transfers dialysate while ensuring flow accuracy.

The flow control unit according to an embodiment of the present invention may include a plurality of fluid chambers each having an internal space, chamber pressurizing members compressing or expanding the internal spaces of the chambers, and a chamber pressurizing member driver operating the chamber pressurizing members. In particular, the flow control unit is configured to include first and second fluid chambers each connected with an inflow tube and an outflow tube, where a fluid is provided to the chamber through the inflow tube and the fluid of the chamber is discharged through the outflow tube. In addition, the outflow tube may be connected to an upper portion of the chamber to allow dialysate to flow upward as it exits.

The flow control unit may further comprise first and second volume chambers. The first volume chamber is positioned downstream of the first chamber, allowing it to hold dialysate flowing from the first chamber. The second volume chamber is placed before the second chamber, enabling it to temporarily store dialysate from the blood dialyzing filter before supplying it to the second chamber.

Unlike the first and second chambers, the first volume chamber may be connected with a single tube through which dialysate flows in and out. The first volume chamber is connected to the dialysate inlet tube, which links the flow control unit and the blood dialyzing filter, upstream of the blood dialyzing filter.

The flow control unit according to an embodiment of the present invention is not limited thereto, and may be modified into other configurations.

For example, the flow control unit is configured to include first and second fluid containers, each having an internal space to accommodate fluid, and a load cell to measure the weight of the fluid containers. The first fluid container holds dialysate upstream of the blood dialyzing filter, while the second fluid container contains dialysate that has passed through the blood dialyzing filter. Here, the load cell may further include a first load cell to measure the weight of the first fluid container and a second load cell to measure the weight of the second fluid container. An additional load cell may be installed to measure the weight of a buffer tank that is placed upstream of the first fluid container and provides dialysate to the first fluid container.

Each fluid container has multiple fluid ports, with flow tubes connected to these ports, enabling dialysate to flow through the containers. For example, the first fluid container is connected to a first container inflow tube for dialysate to enter, a first container outflow tube for dialysate to exit, and a first container vent tube for dialysate or air flow. Similarly, the second fluid container is connected to a second container inflow tube, a second container outflow tube, and a second container vent tube, allowing dialysate and air to flow in and out.

To ensure smooth dialysate flow through the first and second fluid containers, the flow control unit may be equipped with fluid pumps. A first fluid pump transfers dialysate through the first fluid container, and a second fluid pump transfers dialysate through the second fluid container. In other words, the operation of the first fluid pump causes dialysate to flow into and out of the first fluid container. Similarly, the operation of the second fluid pump produces dialysate to flow into and out of the second fluid container.

The method of operating the flow control unit according to an embodiment of the present invention may be embodied to include steps S101 to S108, constituting an operational cycle.

Steps S101 and S102 involve valve operations. In step S101, the first chamber outflow valve V32 is closed (S101A), and the second chamber inflow valve V33 is closed (S101B). In step S102, the first chamber inflow valve V31 and the second chamber outflow valve V34 are opened. That is, steps S101 and S102 each include sub-steps S101A and S101B, and S102A and S102B, respectively.

Step S103 involves the movement of the chamber pressurizing members to expand the internal space of the first chamber (S103A) and compress the internal space of the second chamber (S103B). As the first chamber expands, dialysate flows into the first chamber. With valve V31 open and valve V32 closed, dialysate from the buffer tank is supplied to the first chamber, preventing any retrograde flow from the blood dialyzing filter. Simultaneously, the compression of the second chamber discharges its dialysate. With valve V33 closed and valve V34 open, the dialysate is drained out from the blood dialyzing apparatus, ensuring no backward flow towards the blood dialyzing filter from the second chamber.

Here, the operation may be configured to include a time pause to allow the chambers to reach equilibrium after expansion and compression. This downtime (DT) allows the hydraulic pressures inside the chambers to stabilize within a preset range.

The method of operating the flow control unit is not limited to including step S104. This step may be omitted or significantly shortened to ensure that one operational cycle is completed within a permissible time.

The description for steps S101 and S102 applies similarly to steps S105 and S106, except that the valve operations are reversed.

Step S107 also involves the movement of the chamber pressurizing members, but in this step, the internal space of the first chamber is compressed (S107A) while the second chamber is expanded (S107B). As the first chamber is compressed, its dialysate is supplied to the blood dialyzing filter because valve V31 is closed and valve V32 is open, preventing any backflow toward the buffer tank. Simultaneously, the expansion of the second chamber allows dialysate to flow into it from the blood dialyzing filter due to the opened valve V33 and the closed valve V34. In essence, the description for step S103 applies to step S107, except that the compression and expansion of chambers are reversed.

Since the chambers are being compressed and expanded, respectively, it is advantageous to allow a certain amount of time for the fluid inside the chambers to reach equilibrium. This pause helps achieve more stable pressure conditions inside the chambers.

As mentioned, steps S101 to S108 form a complete operational cycle for the flow control unit. The blood dialyzing apparatus according to an embodiment of the present invention may repeat this cycle. During step S107, the first chamber supplies dialysate to the blood dialyzing filter, while the second chamber removes dialysate from it. During step S103, the first chamber is filled with dialysate, and the second chamber discharges dialysate.

Throughout the cycle, the chambers are either expanded or compressed. The internal volume of the chamber that is expanded or compressed per cycle is referred to as the ‘stroke volume (SV)’ of the cycle. For example, the first chamber expands and compresses during a cycle, and the volume of dialysate that fills and empties the first chamber is the SV of the first chamber (SV51a). Similarly, the second chamber compresses and expands during a cycle, with used dialysate filling the second chamber. The dialysate volume filling the second chamber during that cycle is the SV of the second chamber (SV51b).

Disclosed is the blood dialyzing apparatus according to an embodiment of the present invention, in which multiple fluid chambers are compressed and expanded to transfer dialysate. The multiple chambers ensure dialysate flow rates upstream and downstream of the blood dialyzing filter to be regulated independently or precisely. Neither a separate ultrafiltration pump, nor a separate balancing chamber are required. Therefore, the entire system can be sufficiently miniaturized and light-weighted, and easy to be installed while reducing the cost for blood dialyzing treatment. The blood dialyzing apparatus will eventually be an optimal alternative for the blood dialyzing treatment in a place out of hospitals.

### BRIEF DESCRIPTION OF THE DRAWINGS

The accompanying drawings are included to provide a further understanding of the present invention and are incorporated in and constitute a part of this specification. The drawings illustrate exemplary embodiments of the present invention and, together with the description, serve to explain principles of the present invention. In the drawings:

FIG. 1 is a view illustrating a schematic diagram of a blood dialyzing apparatus according to an embodiment of the present invention;

FIG. 2 is a view illustrating a blood dialyzing filter according to an embodiment of the present invention;

FIG. 3 is a view illustrating a circuit diagram of a blood dialyzing apparatus according to an embodiment of the present invention;

FIG. 4 is a view illustrating a circuit diagram of a blood dialyzing apparatus having a flow control unit according to an embodiment of the present invention;

FIG. 5 is a detailed view illustrating first and second chambers according to an embodiment of the present invention;

FIG. 6 is a view illustrating a pressurizing clamp valve according to an embodiment of the present invention;

FIG. 7 is a flowchart illustrating steps of operating a flow control unit having a first chamber and a second chamber according to an embodiment of the present invention;

FIGS. 8A and 8B are views illustrating schematic diagrams of an operational sequence of a flow control unit having first and second chambers according to an embodiment of the present invention;

FIGS. 9A and 9B are views illustrating schematic diagrams of operational sequence of a flow control unit having first and second chambers according to an embodiment of the present invention;

FIG. 10 is a view illustrating a circuit diagram of a blood dialyzing apparatus having a flow control unit according to an embodiment of the present invention;

FIGS. 11A and 11B are flowcharts illustrating steps of operating first and second fluid chambers and volume chambers according to an embodiment of the present invention;

FIGS. 12A to 12C are views illustrating schematic diagrams of a modified operational sequence of first and second fluid chambers and volume chambers according to an embodiment of the present invention;

FIG. 13 is a view illustrating a circuit diagram of a blood dialyzing apparatus having a flow control unit according to an embodiment of the present invention;

FIG. 14 is an enlarged view illustrating first and second fluid containers according to an embodiment of the present invention;

FIGS. 15 to 17 are flowcharts illustrating steps of operating a flow control unit where a first fluid container and a second fluid container are separately provided according to an embodiment of the present invention;

FIGS. 18A and 18B are views illustrating schematic diagrams of various operational sequences of a flow control unit having a first fluid container and a second fluid container according to an embodiment of the present invention;

FIG. 19 is a view illustrating a circuit diagram of a blood dialyzing apparatus equipped with a flow control unit according to an embodiment of the present invention;

FIG. 20 is an enlarged view illustrating first and second fluid containers which are stacked vertically with each other according to an embodiment of the present invention;

FIG. 21 is a view illustrating a circuit diagram of a blood dialyzing apparatus according to another embodiment of the present invention;

FIG. 22 is a flowchart illustrating modified steps of operating a flow control unit having first and second fluid containers according to an embodiment of the present invention;

FIGS. 23A to 23F are views illustrating circuit diagrams representing steps of operating a flow control unit shown in FIG. 19 according to an embodiment of the present invention;

FIGS. 24A to 24C are views illustrating schematic diagrams of operational sequences of a flow control unit according to an embodiment of the present invention;

FIG. 25 is a view illustrating a circuit diagram of a blood dialyzing apparatus according to another embodiment of the present invention; and

FIGS. 26A and 26B are views illustrating a method of calibrating a flow control unit having a load cell according to an embodiment of the present invention.

### DETAILED DESCRIPTION OF PREFERRED EMBODIMENTS

Preferred embodiments of the present invention will be described below in more detail with reference to the accompanying drawings. The present invention may, however, be embodied in different forms and should not be constructed as limited to the embodiments set forth herein. Elements and components disclosed in the drawings may be exaggerated or simplified to improve the clarity and convenience of the description. Terms or languages defined in the present disclosure may have different meaning according to the users' intention or practice. These terms should be interpreted as a meaning corresponding to the technical concept of the present invention disclosed throughout the specification of the present invention.

As used herein, the singular forms “a,” “an,” and “the” are intended to include the plural forms as well, unless the context clearly indicates otherwise. In addition, the expressions defining the relationship of elements or components should be interpreted as broad as possible. For example, it will be understood that when an element or layer is referred to as being “on,” “connected to,” “coupled to,” or “adjacent to” another element or layer, it can be directly on, connected, coupled, or adjacent to the other element or layer, or intervening elements or layers may be present therebetween. It will also be understood that when an element is same or identical to another element, the element can be completely same or identical to another element, or it includes that the two elements may be “substantially” similar to each other. In the same manner, for the expression showing the equivalence of time such as “simultaneously” or “at the same time,” it should be understood that it happens completely at the same time, or they may happen at substantially the similar time. The same reference denotations may be used to refer to the same or substantially the same elements throughout the specification and the drawings.

Hereinafter, the blood dialyzing apparatus will be described in detail with reference to the accompanying drawings.

The blood dialyzing apparatus may be configured to comprise a blood dialyzing device 2 and a disposable set 3. The blood dialyzing device is a hardware unit with a case in which various electric elements are mounted to perform the treatment. Software and programming to run the electric elements are installed. The disposable set is a consumable element used for each treatment. For example, the disposable unit includes tubes through which blood, dialysate, or any biologic fluid flows, air drip chambers to remove air bubbles, and/or a blood dialyzing filter.

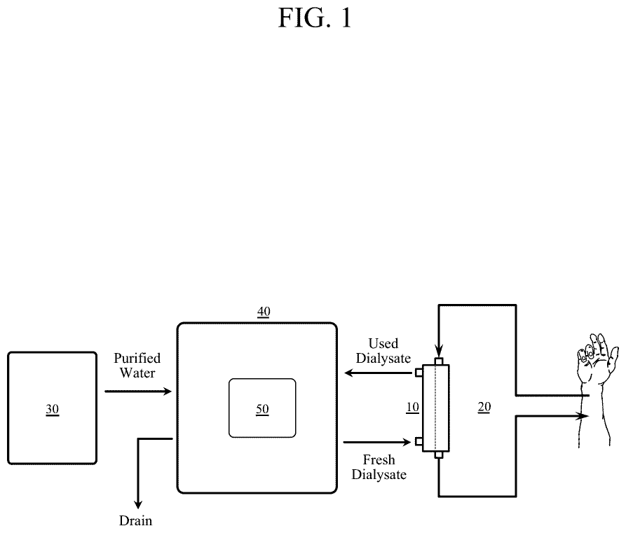

FIG. 1 is a schematic diagram of a blood dialyzing apparatus according to an embodiment of the present invention. The blood dialyzing apparatus 1 preferably includes a blood dialyzing filter 10, where blood is dialyzed, a blood flowing unit 20 through which blood is supplied to the blood dialyzing filter 10 and returned to the patient, and a dialysate flowing unit 40 where dialysate is prepared and transferred through a blood dialyzing filter 10. The dialysate flowing unit 40 may further include a flow control unit 50 that transfers dialysate while ensuring flow accuracy.

The blood dialyzing apparatus 1 also includes a water treatment unit 30, which generates purified water through a reverse osmosis (RO) process and supplies it to the dialysate flowing unit 40. The water treatment unit 30 may include multiple filtration stages, such as a pre-processing filter, a carbon filter, a reverse osmosis filter, ion-exchange resin beds, and an endotoxin retention filter. The water treatment unit 30 can be embodied into a different configuration to prepare ultrapure water that meets the requirements of blood dialyzing treatment.

An exemplary blood dialyzing apparatus may include devices for preserving blood, separating blood cells or plasma from whole blood, dialyzing blood of patients with renal failure (acute or chronic), or detoxifying blood for patients with liver failure (acute or acute-on-chronic) or multi-organ failure. In other words, any device that replaces impaired functions of the lungs, heart, liver, or kidneys may be used as the blood dialyzing apparatus according to an embodiment of the present invention.

In this specification, the term ‘dialysate’ is used. However, it should not be limited to the fluid used for hemodialysis, continuous renal replacement therapy (CRRT), or peritoneal dialysis. Dialysate can refer to any fluid used in treatments requiring extracorporeal blood circulation, such as plasma, serum, distilled water, isotonic saline solution, lactose solution, and similar fluids. Additionally, throughout the specification, the term ‘dialysate’ is used to refer to a fluid or fluidic solution.

### Blood Dialyzing Filter

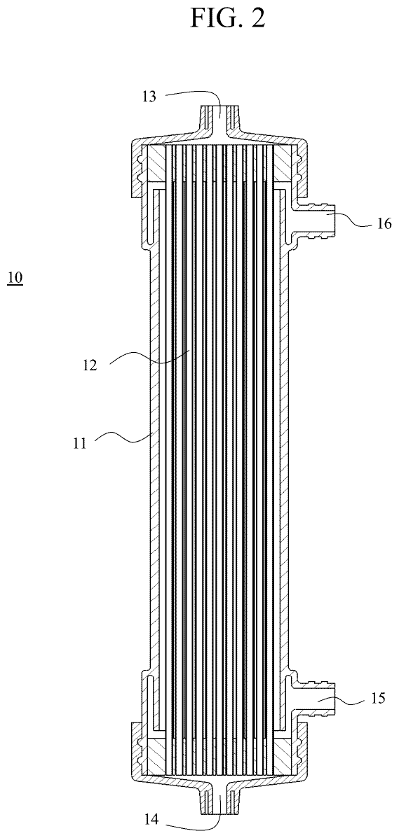

The blood dialyzing filter 10 includes various filter apparatuses to dialyze a patient's blood. Referring to FIG. 2, the blood dialyzing filter 10 may include a blood dialyzing membrane 12 housed within a filter housing 11. The internal space of the filter housing 11 can be divided into multiple flow regions by the membrane 12, each through which a separate fluid flows. In an embodiment, the blood dialyzing filter 10 is divided into a blood flow region and a dialysate flow region by the blood dialyzing membrane 12.

The filter housing 11 is provided with a first blood port 13 and a second blood port 14 disposed at an opposite side thereof. Blood may enter the blood dialyzing filter 10 through the first blood port 13 and leave therefrom through the second blood port 14. Blood tubes 21 and 22 may be connected to the blood ports 13 and 14, respectively, to allow blood to flow through blood dialyzing filter 10. Also, a first dialysate port 15 and a second dialysate port 16 may be provided on the filter housing 11 to allow the dialysate to flow through the blood dialyzing filter 10. Specifically, dialysate may be provided to the blood dialyzing filter 10 through the first dialysate port 15 and is discharged therefrom through the second dialysate port 16.

Blood passes through the blood flow region inside the blood dialyzing filter 10 and dialysate passes through the dialysate flow region. Blood and dialysate may be desirably configured to flow in the opposite directions to each other. The blood dialyzing filter 10 is not limited to the structure shown in the drawing, and may be modified into other forms including a hemodialyzer, an adsorption filter column, or a hemodiafilter.

### Blood Flowing Circuit

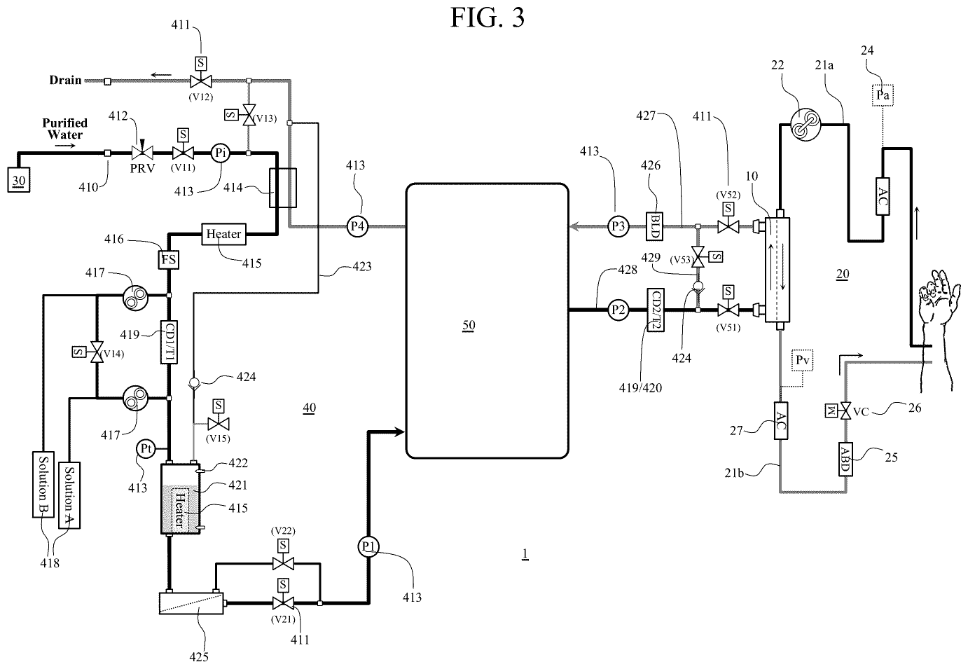

Blood flows through the blood flowing unit 20. As shown in FIG. 3, blood withdrawn from a patient is supplied to the blood dialyzing filter 10 through an arterial blood flowing tube 21a. In the blood dialyzing filter 10, mass transfer occurs between the blood and the dialysate. Blood that has passed through the blood dialyzing filter 10 returns to the patient through the venous blood flowing tube 21b. To facilitate this process, the blood flowing unit 20 may be provided with a blood pump 22 to transfer blood through the arterial and venous blood flowing tubes 21a and 21b.

Air chambers are mounted on the blood flowing tubes 21a and 21b to remove air bubbles from the bloodstream. Various safety sensors are also mounted throughout the blood flowing unit 20. For example, pressure sensing components are provided to measure hydraulic blood pressures in the arterial blood flowing tube 21a (Pa) and the venous blood flowing tube 21b (Pv). The Pa and Pv values can provide important safety information to ensure safe blood dialyzing treatment, including but not limited to whether an adequate amount of blood is supplied to the blood dialyzing filter 10, whether an arterial or venous needle is clogged, or whether the blood dialyzing filter 10 is coagulated.

Additionally, it is crucial to prevent air bubbles from returning to the patient. For this purpose, the blood flowing unit 20 may be equipped with an air bubble detector 25, preferably positioned downstream of the venous air chamber 27. A venous clamp valve 26 can also be installed downstream of the air bubble detector 25 in the venous blood flowing tube 21b to prevent air bubbles from entering the patient.

The circuit diagram of the blood flowing unit 20 shown in the drawing is merely exemplary. It is evident to those of ordinary skill in the art that the flowing circuit of the blood flowing unit 20 can be modified as needed to meet the objectives of the blood dialyzing treatments.

### Dialysate Flowing Unit

FIG. 3 illustrates a circuit diagram of the blood dialyzing apparatus 1 according to an embodiment of the present invention. Purified water prepared by the water treatment unit 30 is supplied to the dialysate flowing unit 40 through an inlet connector 410. The purified water is then depressurized to a predetermined hydraulic pressure range, such as 0.1 to 3 bar, preferably 0.5 to 2 bar, by passing through a pressure regulating valve (PRV) 412.

Flow control valves 411 are installed throughout the dialysate flowing unit 40 to regulate the flow passages through the tubes. Exemplary valves include on-off valves, solenoid valves, metering valves, clamp valves, motor-driven clamp valves, and the like. According to various embodiments, the valves 411 may be automated and controlled by electrical signals driven by a predetermined operation protocol, such as those programmed into a PCB circuit and controlled by firmware. The valves are labeled as V, ranging from V11 to V65.

Various safety and monitoring sensors are also provided throughout the dialysate flowing unit 40. For example, pressure sensors 413 are installed throughout the unit, as illustrated in FIG. 3. These pressure sensors, labeled as P (e.g., Pi, Pt, P0, P1 to P4), measure the hydraulic pressures of purified water or dialysate. As mentioned earlier, pressure sensing points such as Pa and Pv are provided to measure the blood pressures in the blood flowing unit 20.

The dialysate may be adjusted to a temperature similar to human body temperature before being supplied to the blood dialyzing filter 10. To achieve this, the blood dialyzing apparatus 1 may include a heat exchanger 414 and a heater 415. The heat exchanger 414 recovers heat from the used dialysate and transfers it to the incoming RO water. Additionally, the heater 415 adjusts the temperature of the RO water to a desired level. A flow sensor 416 may also be installed, either upstream or downstream of the heater 415, to measure the flow rate through the heater and ensure proper water flow.

Fresh dialysate is produced by mixing purified RO water with various ion concentrate solutions, which is then transferred to the blood dialyzing filter 10. For example, acid and bicarbonate concentrate solutions 418 (or acid and bicarbonate powder) may be mixed with RO water to produce fresh dialysate. This process adjusts the ion concentrations, such as bicarbonate and sodium, and the pH of the dialysate. To facilitate this, dialysate processing pumps 417 may be required to transfer the acid and/or bicarbonate concentrate solutions 418. Since the dialysate processing pump 417 needs to deliver precise amounts of concentrate solution 418, a precise metering pump may be preferred. Exemplary dialysate processing pumps 417 include rotary piston pumps, peristaltic pumps, piston pumps, and the like. The dialysate processing pumps 417 can be divided into first and second pumps to separately transfer acid and bicarbonate concentrate solutions 418.

A fresh dialysate container and a used dialysate container, not shown in the drawings, may be used to store fresh dialysate or collect used dialysate, respectively. However, fresh dialysate can be supplied to the blood dialyzing filter 10 without being stored in the fresh dialysate container, and used dialysate may be discarded without being collected in the used dialysate container.

It is crucial to ensure that the RO water is mixed with the predetermined amounts of concentrate solutions 418 to maintain the purity of the dialysate. Any error in the mixing ratio can alter the osmotic and pH conditions of the dialysate, potentially resulting in blood cell damage when the dialysate interacts with blood in the blood dialyzing filter 10. Therefore, the dialysate flowing unit 40 is preferably equipped with a conductivity sensor 419, such as CD1 or CD2. In FIG. 3, CD1 measures the conductivity of the RO water and B-sol mixture, while CD2 measures the conductivity of the dialysate before it is supplied to the blood dialyzing filter 10. To account for the effect of temperature on conductivity, temperature sensors 420 are also installed to measure the temperatures of the fluid.

The dialysate is not limited to being produced through the dialysate flowing unit 40; it may also be provided using a pre-made dialysate bag.

A buffer tank 421 is positioned downstream of the dialysate processing portion. The buffer tank 421 stores the produced dialysate while also degassing it. To facilitate this, a vent port is provided at the upper side of the buffer tank 421, connected to a vent line 423, allowing air to be removed from or supplied to the buffer tank. An additional pressure sensor 413, labeled Pt, monitors the internal pressure of the buffer tank 421, while a level sensor 422 detects the fluid level inside the tank. According to an embodiment of the present invention, the buffer tank 421 may also include a heater 415 to control the dialysate temperature.

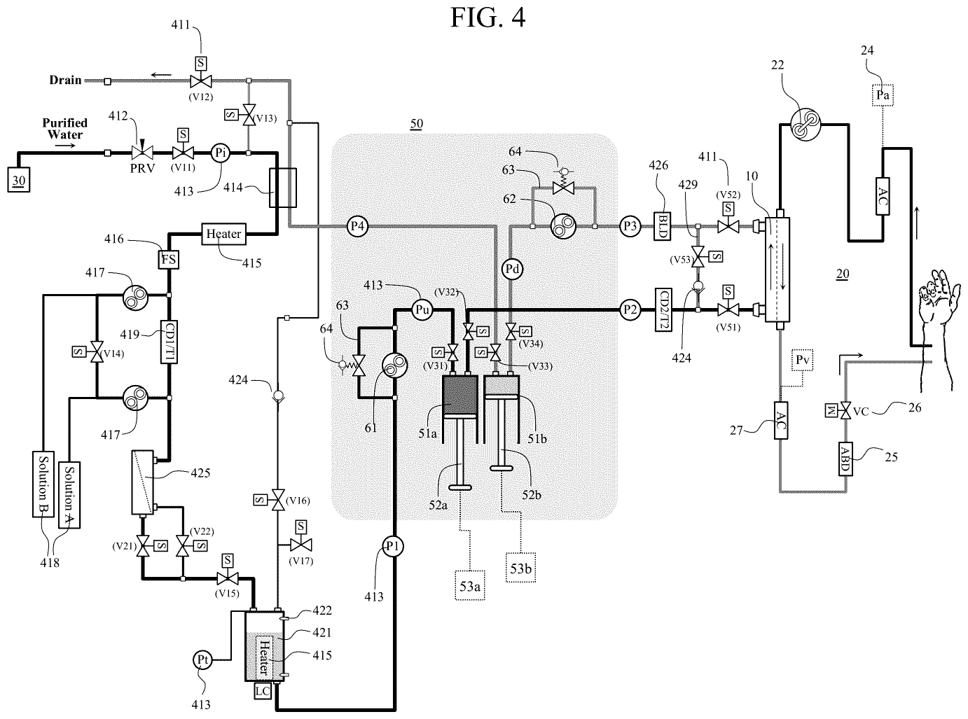

The dialysate flowing unit 40 of the blood dialyzing apparatus 1, according to an embodiment of the present invention, may also be embodied to include an endotoxin filter 425. This filter removes any pyrogenic substances from the dialysate. In FIG. 3, the endotoxin filter 425 is placed downstream of the buffer tank 421. Alternatively, as shown in FIG. 4, the endotoxin filter 425 can be installed upstream of the buffer tank 421 or downstream of the flow control unit 50 in the dialysate inlet tube 427, which connects the flow control unit 50 to the blood dialyzing filter 10. It is evident to those skilled in the art that the position of the endotoxin filter 425 can be modified as needed to ensure harmful substances are prevented from entering the blood dialyzing filter 10.

A flow control unit 50 is provided to supply dialysate to and remove used dialysate from the blood dialyzing filter 10. The flow control unit 50 also regulates the flow rates of the dialysate, such as milliliters per minute, both upstream (before) and downstream (after) of the blood dialyzing filter 10. For example, the flow control unit 50 can maintain the difference in dialysate flow rates upstream and downstream of the blood dialyzing filter 10 within a predetermined permissible range.

Additional safety measures are included downstream of the flow control unit 50. For instance, a second conductivity sensor 419 (CD2) and an additional temperature sensor 420 may be installed before the blood dialyzing filter 10 to ensure that the dialysate's quality and temperature meet required standards. Since mass transfer between blood and dialysate occurs within the blood dialyzing filter 10, the dialysate temperature may be adjusted to closely match body temperature. According to an embodiment of the present invention, the temperature (T2) may be maintained between 32° C. and 39° C., and more preferably between 35.5° C. and 37.5° C.

Additional pressure sensors 413 are installed to measure the hydraulic pressures of the dialysate upstream and downstream of the blood dialyzing filter 10, denoted as P2 and P3 in FIG. 3. The average value of P2 and P3 is often used as the mean dialysate pressure of the blood dialyzing filter 10, such as when calculating the transmembrane pressure (TMP).

Furthermore, the blood dialyzing apparatus 1 according to an embodiment of the present invention includes a blood leak detector 426 to sense whether blood leakage occurs in the blood dialyzing filter 10. The blood leak detector 426 detects blood cell damage or hemolysis in the dialysate that has passed through the blood dialyzing filter 10 and may be positioned at the dialysate outlet tube 428 of the blood dialyzing filter 10.

If the dialysate quality, such as conductivity or temperature, does not meet the requirements for blood dialyzing treatment, the dialysate should be prevented from being supplied to the blood dialyzing filter 10. For this purpose, a dialysate bypass tube 429 is provided, connecting the dialysate inlet tube 427 (connected to the first dialysate port 15) and the dialysate outlet tube 428 (connected to the second dialysate port 16) of the blood dialyzing filter 10. Additionally, a filter bypass valve V53 may be disposed in the dialysate bypass tube 429 to regulate the flow. Similarly, flow control valves 411 are installed on the dialysate inlet tube 427 (V51) and the dialysate outlet tube 428 (V52).

### Flow Control Unit

Hereinafter, the flow control unit 50 and its operational steps will be described in detail with reference to the accompanying drawings.

FIG. 4 illustrates a circuit diagram of the blood dialyzing apparatus 1 with the flow control unit 50 according to an embodiment of the present invention. The flow control unit 50 may be configured to include multiple fluid chambers 51, each having an internal space, a chamber pressurizing member 52 to compress or expand the internal spaces of the fluid chambers, and a chamber pressurizing member driver 53 to operate the chamber pressurizing member 52.

Specifically, the flow control unit 50 in FIG. 4 includes first and second fluid chambers 51a and 51b, each compressed and expanded by chamber pressurizing members 52a and 52b, respectively located inside each fluid chamber. The first and second chamber pressurizing members 52a and 52b can be independently driven by separate chamber pressurizing member drivers 53a and 53b.

Each fluid chamber 51a and 51b is connected to inflow and outflow tubes. The first chamber 51a is connected to the first chamber inflow tube 55a and the first chamber outflow tube 55b. Dialysate flows into the first chamber 51a through the inflow tube 55a and exits through the outflow tube 55b. Similarly, the second chamber 51b is connected to the second chamber inflow tube 55c and the second chamber outflow tube 55d. The outflow tube may be connected to an upper portion of the chamber to allow dialysate to flow upward as it flows out.

The terms “inflow” and “outflow” tubes are used to describe the tubes connected to the chamber, but they should not be interpreted to mean that fluid must enter the chamber through the inflow tube or exit through the outflow tube. For example, fluid can flow into the chamber through the outflow tube, or it can be provided to or discharged from the chamber through both the inflow and outflow tubes. Additionally, each chamber may be connected to both inflow and outflow tubes, but they may overlap in such a way that a single tube is connected to the chamber.

The chamber pressurizing member driver 53 includes various structures that enable the chamber pressurizing members to move along a straight or curved line to compress or expand the internal spaces of the chambers. An exemplary chamber pressurizing member driver may include a cam that pushes the chamber pressurizing member in a rectilinear direction and a motor that rotates the cam. Alternatively, the chamber pressurizing member driver may include a motor, a circular gear rotated by the motor, and a linear gear that moves along a straight line due to the rotation of the circular gear. As the cam or circular gear rotates, the chamber pressurizing members move in a rectilinear direction. When the motor rotates further or in the opposite direction, the chamber pressurizing members may move in the opposite direction.

In addition, the flow control unit 50 according to an embodiment of the present invention may further comprise fluid pumps to transfer dialysate to the first and second chambers 51a and 51b. A first chamber pump 61 may be provided upstream of the first chamber 51a to supply dialysate to the first chamber 51a. A second chamber pump 62 may be disposed to pump dialysate from the blood dialyzing filter 10 to the second chamber 51b.

The fluid pumps 61 and 62 are illustrated as gear pumps in the drawings, but they are not limited to this type. Any type of volume displacement pump may be used, including but not limited to peristaltic pumps, lobe pumps, rotary piston pumps, or piston pumps.

Additionally, a bypass tube 63 connecting the upstream and downstream sides of each flow pump 61 or 62 may be placed around the pump. To maintain a predetermined pressure level at the outlet of the pump 61 or 62, a relief valve 64 may be installed in the bypass tube 63.

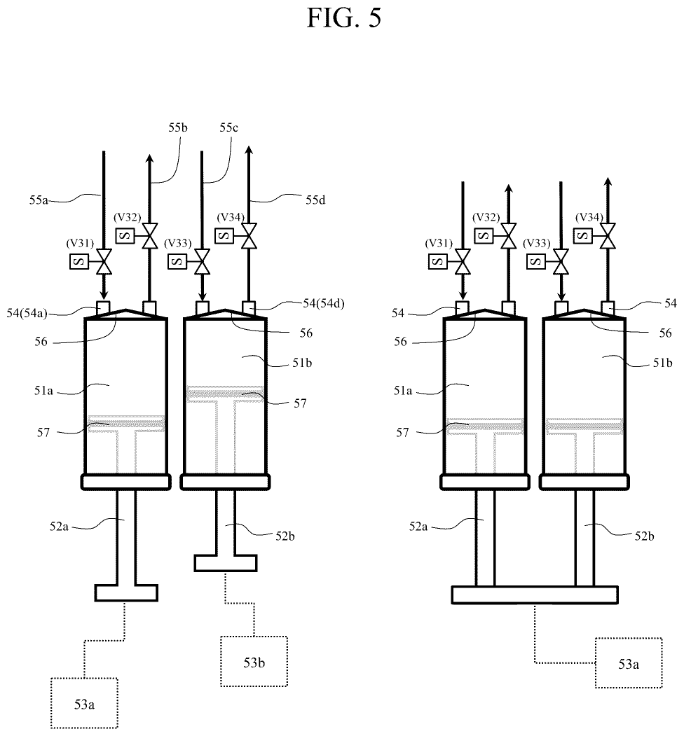

As mentioned above, various flow control mechanisms are provided throughout the dialysate flowing unit 40 to regulate flow passages through the tubes. Flow control valves 411 may be disposed on the inflow and outflow tubes of the first and second chambers 51a and 51b. Referring to FIG. 5, a flow control valve V31 is installed in the first chamber inflow tube 55a, which opens or closes the flow passage through tube 55a. Similarly, the flow control valve V32 is installed in the first chamber outflow tube 55b to regulate flow through tube 55b. Likewise, the flow control valves V33 and V34 are installed on tubes 55c and 55d to control the flow through these tubes.

Solenoid valves (S) are depicted in the drawings. These solenoid valves are automated and controlled by electrical signals driven by a predetermined operation protocol, such as that programmed in a PCB circuit and by firmware coding. However, the flow control valves according to an embodiment of the present invention are not limited to the solenoid valves and can be various other types of valves that can open or close the flow passages through the tubes, including but not limited to one-way valves, on-off valves, pressurizing clamp valves, rotating type clamp valves, pneumatic valves, or a combination of these valves. One-way valves ensure fluid flows in one direction while solenoid valves and on-off valves open or shut off the flow. Pneumatic valves, comprising a pneumatic driver and a pneumatic channel, compress or decompress, thereby blocking or opening the flow tubes 55a to 55d using pneumatic pressure.

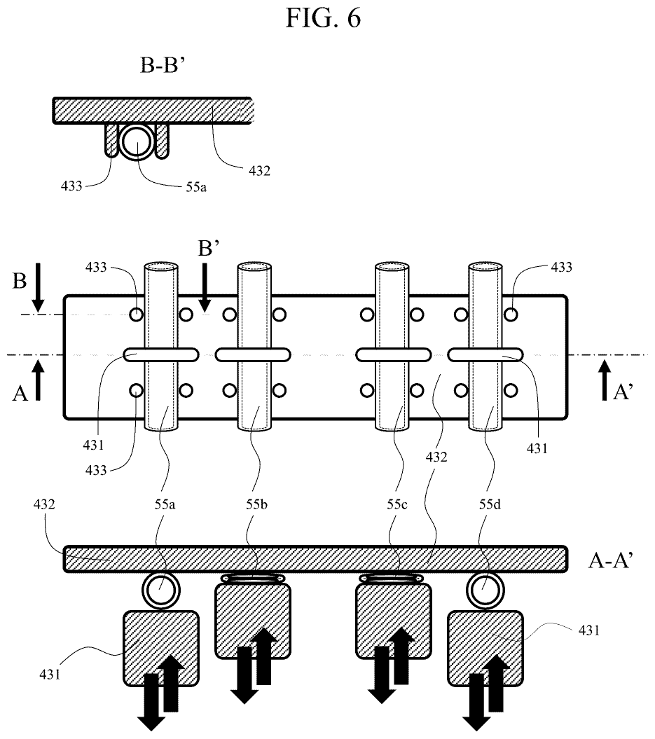

FIG. 6 is a view illustrating a schematic view of the pressurizing tube clamp valve according to an embodiment of the present invention. The pressurizing tube clamp valve may include a tube compressing member 431 that reciprocates in a straight line to compress a portion of the tubes 55a to 55d, thereby blocking the flow passage, a tube support wall 432 supporting the tubes compressed by the tube compressing member 431, and a flow blocking member driver providing a straight or curved force to the tube compressing member 431. When the tube compressing member 431 moves towards the tubes, its end compresses the tubes against the tube support wall 432, blocking the flow. The tubes can be firmly fixed in place by a tube holder 433.

When the tubes are made of flexible materials, such as rubber, silicone, polyurethane, polyacetate, or other polymers, it is possible to bend the flow tubes at a predetermined angle to block the flow passage. The tube compressing member 431 may be embodied to include various structures to bend the tubes to block the flow.

The flow blocking member driver includes various structures that can apply a reciprocating movement force (that is, for a rectilinear or curvilinear movement) to the tube compressing member 431. Substantially the same description for the chamber pressurizing member driver 53 can be applied to the flow blocking member driver. For example, the flow blocking member driver may include a cam for pushing the tube compressing member 431 toward the tube support wall 432 supporting the tubes and a motor rotating the cam. When the tube compressing member 431 compresses the tubes due to the rotation of the cam, the flow therethrough may be blocked. When an external force by the cam is removed, the tube compressing member 431 detaches from the tube, and the tube may be restored to the original state, expanding the inside of the tube. Alternatively, an eccentric cam connected to a motor may rotate and compress one side of the tube and block the flow therethrough. The cam further rotates such that an external force applied by the cam may be removed and the tube is restored to its original status, expanding the inside of the tube.

The chambers 51a to 51d may be configured with a cylindrical internal space, while the chamber pressurizing members 52a to 52d have a piston shape, reciprocally and detachably disposed inside the cylindrical chambers, as depicted in FIG. 5. The chambers 51a to 51d can be made from a substantially inflexible material with a predetermined shape, such as plastic, polycarbonate, polyurethane, or metal. The chamber pressurizing members 52a to 52d may preferably include a portion 57 made of a substantially flexible material, such as rubber, polymer, or silicone.

However, the structure of the chamber and the chamber pressurizing member is not limited to this configuration. Any container with an internal space to accommodate a fluid and any means to pressurize or expand the internal space to allow fluid flow can be used as the chamber and the chamber pressurizing member. Examples of such chambers include fluid sacs, fluid bags, or fluid tubes that are flexible. Any mechanism that pressurizes or expands these flexible containers can be used as the chamber pressurizing member. In this case, the chamber pressurizing member may preferably have an inflexible portion to compress the flexible chambers.

Additionally, the fluid ports 54a to 54d may be placed at the upper portion of the chambers 51a to 51d along their longitudinal direction. This placement facilitates the removal of air bubbles from the chambers when the fluid is discharged. To aid this process, the chambers 51a to 51d may further include an oblique part 56 on their upper surface, as shown in FIG. 5.

Hereinafter, a method of operating the flow control unit 50 according to an embodiment of the present invention will be described in detail with reference to the accompanying drawings.

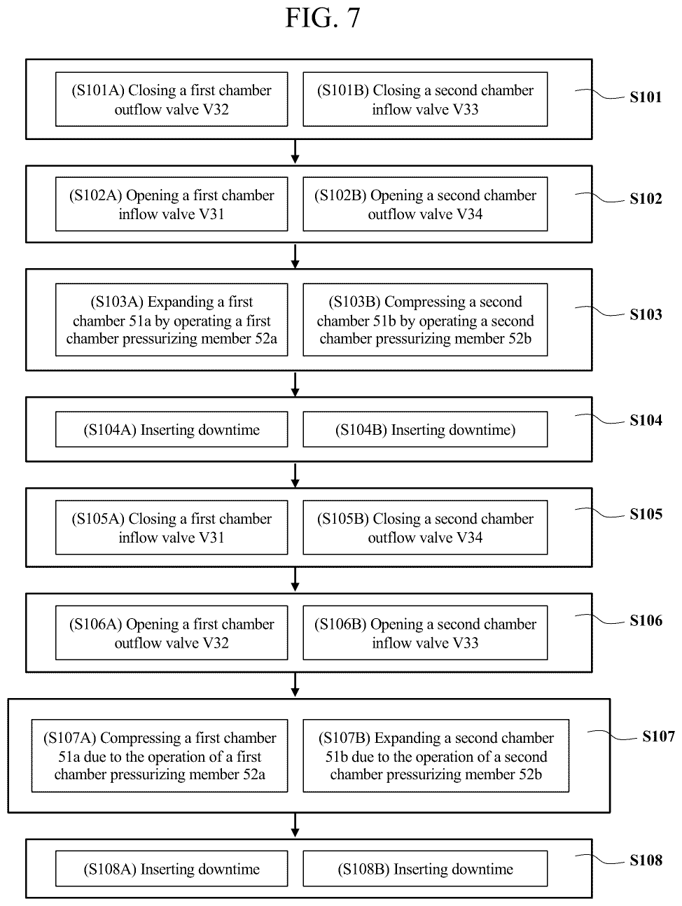

FIG. 7 is a flowchart illustrating the steps of operating the flow control unit 50, which includes the first chamber 51a and the second chamber 51b. The steps, S101 to S108, constitute an operational cycle.

### (1) S101 and S102

Steps S101 and S102 involve valve operations. In step S101, the first chamber outflow valve V32 is closed (S101A), and the second chamber inflow valve V33 is closed (S101B). In step S102, the first chamber inflow valve V31 and the second chamber outflow valve V34 are opened. Steps S101 and S102 each include sub-steps S101A and S101B, and S102A and S102B, respectively, as shown in FIG. 7. Here, ‘A’ in the sub-step names relates with the operation of the first chamber 51a, while ‘B’ indicates the operation of the second chamber 51b.

### (2) S103

Step S103 involves the movement of the chamber pressurizing members 52a and 52b to expand the internal space of the first chamber 51a (S103A) and compress the internal space of the second chamber 51b (S103B). As the first chamber 51a expands, dialysate flows into the first chamber 51a. With valve V31 open and valve V32 closed, dialysate from the buffer tank 421 is supplied to the first chamber 51a, preventing any retrograde flow from the blood dialyzing filter 10. Simultaneously, the compression of the second chamber 51b discharges its dialysate. With valve V33 closed and valve V34 open, the dialysate is drained out from the blood dialyzing apparatus 1, ensuring no backward flow towards the blood dialyzing filter 10 from the second chamber 51b.

### (3) S104

The operation of the flow control unit 50 may include a time pause to allow the chambers to reach equilibrium after expansion and compression. This downtime (DT) allows the hydraulic pressures inside the chambers to stabilize within a preset range. The duration for S104, which is the time delay after S103, can be set between 0.2 and 6.8 seconds, preferably between 0.8 and 3.6 seconds.

However, the method of operating the flow control unit 50 according to an embodiment of the present invention is not limited to including step S104. This step may be omitted or significantly shortened to ensure that one operational cycle is completed within a permissible time.

### (4) S105 and S106

The description for steps S101 and S102 applies similarly to steps S105 and S106, except that the valve operations are reversed. Essentially, the same process described for steps S101 and S102 can be applied to steps S105 and S106.

### (5) S107

Step S107 also involves the movement of the chamber pressurizing members 52a and 52b, but in this step, the internal space of the first chamber 51a is compressed (S107A) while the second chamber 51b is expanded (S107B). As the first chamber 51a is compressed, its dialysate is supplied to the blood dialyzing filter 10 because valve V31 is closed and valve V32 is open, preventing any backflow toward the buffer tank 421. Simultaneously, the expansion of the second chamber 51b allows dialysate to flow into it from the blood dialyzing filter 10 due to the opened valve V33 and the closed valve V34. In essence, the description for step S103 applies to step S107, except that the compression and expansion of chambers 51a and 51b are reversed.

### (6) S108

Since chambers 51a and 51b are being compressed and expanded, respectively, it may be advantageous to allow a certain amount of time for the fluid inside the chambers to reach equilibrium. This pause helps achieve more stable pressure conditions inside the chambers.

As mentioned, steps S101 to S108 form a complete operational cycle for the flow control unit 50. The blood dialyzing apparatus 1 according to an embodiment of the present invention may be configured to repeat this cycle. During step S107, the first chamber 51a supplies dialysate to the blood dialyzing filter 10, while the second chamber 51b removes dialysate from it. During step S103, the first chamber 51a is filled with dialysate, and the second chamber 51b discharges dialysate.

Here, the terms ‘first’ and ‘second’ chambers are merely used to describe the two chambers. When one chamber is compressed and the other is expanded, it indicates that one chamber is undergoing compression while the other is expanding.

In addition, while the flow control unit 50 typically repeats a cycle of steps S101 to S108, it is not limited to including all of these steps in every cycle. The operational cycle can start at any step and end at a different step. For instance, the cycle may begin at step S105 and end at step S104.

Throughout the cycle, the chambers are either expanded or compressed. The internal volume of the chamber that is expanded or compressed per cycle is referred to as the stroke volume (SV) of the cycle. For example, the first chamber 51a expands and compresses during a cycle, and the volume of dialysate that fills and empties the first chamber 51a is the SV of the first chamber 51a (SV51a). Similarly, the second chamber 5ib compresses and expands during a cycle, with used dialysate filling the second chamber 51b. The dialysate volume filling the second chamber 51b during that cycle is the SV of the second chamber 51b (SV51b).

The flow control unit 50 according to an embodiment allows the SV51a and SV51b per cycle to vary as needed. The difference in these stroke volumes between the first chamber 51a and the second chamber 51b generates water flux across the membranes 12. The blood dialyzing apparatus 1 according to an embodiment of the present invention can regulate the amount of water flux across the membranes 12—either from blood to dialysate (termed ultrafiltration or UF) or from dialysate to blood (termed backfiltration or BF). In other words, the difference in flow rates between the first chamber 51a and the second chamber 51b results in net fluid removal from the patient, referred to as the net UF rate.

For example, the expanded SV51b may be equal to or greater than the compressed SV51a. Specifically, SV51b may be greater than SV51a by 0% to 20%, preferably by 0% to 16%, and more desirably by 0% to 8%. Here, 0% indicates that SV51b is equal to SV51a.

In addition, the flow control unit 50 may be configured to have the SV51a and SV51b values, each ranging between 40 mL and 500 mL per cycle. Preferably, SV51a and SV51b can range between 50 mL and 240 mL per cycle, and more preferably between 60 mL and 140 mL. Furthermore, the difference between SV51a and SV51b, i.e., SV51a−SV51b per cycle, may be set to range from −40 to 28 mL. This means SV51a can be set to a value that is larger than SV51b by 28 mL or smaller than SV51b by 40 mL per cycle. For example, when the second chamber 51b is filled with 100 mL of dialysate during expansion (i.e., SV51b=100 mL), the first chamber 51a may supply 60 mL to 128 mL of dialysate to the blood dialyzing filter 10.

The stroke volumes of the first and second chambers 51a and 51b can vary with each cycle or during a predetermined period of cycles, allowing for the manipulation of net water flux across the dialysis membranes 12. This variation improves mass transfer and dialysis efficiency.

The stroke volumes of the chambers can also be adjusted based on the cycle time, i.e., the duration of each cycle. The cycle time must be determined in consideration of the stroke volumes of the first and second chambers 51a and 51b, as the stroke volumes and cycle time ultimately determine the dialysate flow rate, such as in mL per minute. According to an embodiment, the cycle time may be set between 4.2 and 56 seconds, more preferably between 6.4 and 33 seconds. For example, if the cycle time is set to 12 seconds, the flow control unit 50 will repeat five cycles per minute.

When the chamber is made of a substantially inflexible material with a cylindrical shape and a uniform inner diameter, the stroke volumes of the chamber—whether compressed or expanded-vary according to the distance the chamber pressurizing members 52a and 52b move. For example, the stroke volume can be determined by the chamber radius (R) and the length (d) that the chamber pressurizing member travels, as follows:

$$SV 51 a = π * R {1}^{2} * d 1 \\ SV 51 b = π * R {2}^{2} * d 2$$

Where R1 and R2 are the radii of the chambers 51a and 51b, respectively. The distances d1 and d2 are the rectilinear movements of the chamber pressurizing members 52a and 52b. When R1 and R2 are equal, SV51a and SV51b are determined by d1 and d2.

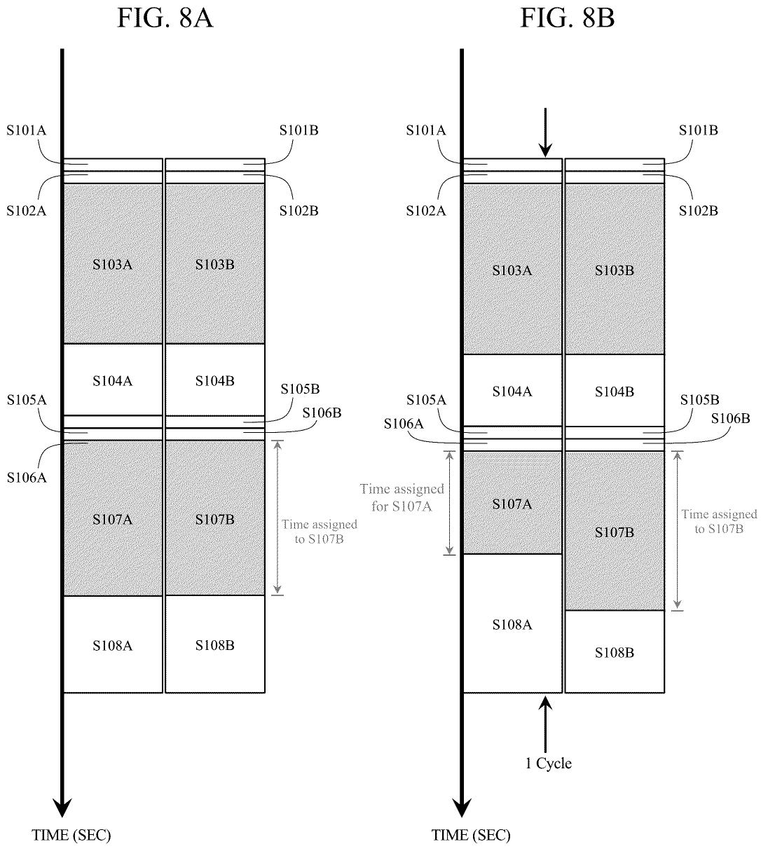

Steps S101A and S101B may take approximately the same amount of time, ranging from 0.2 to 3.2 seconds, and more preferably from 0.2 to 1.6 seconds. This timing is similarly applied to S102A and S102B, S105A and SI05B, and S106A and S106B. Likewise, steps S103 and S107 may take approximately the same amount of time, ranging from 1.5 to 15 seconds, more preferably from 2 to 8 seconds. Additionally, S103A may take roughly the same amount of time as S103B. Similarly, steps S107A and S107B may take almost the same amount of time, as illustrated in FIG. 8A, where the vertical axis represents time in seconds.

However, the operation of the flow control unit 50 according to an embodiment of the present invention is not limited to these specific timings. The starting sequences of the steps and the time assigned to each step can be modified as needed.

As shown in FIG. 8B, for example, step S107A may be shorter than S107B. The compression of the first chamber 51a may take less time than the expansion of the second chamber 51b. Specifically, the compression of the first chamber 51a may take 40% to 80% of the time required for the expansion of the second chamber 51b. For instance, if the expansion of the second chamber 51b takes 6 seconds, the compression of the first chamber 51a may take between 2.4 and 4.8 seconds. Conversely, the expansion of the second chamber 51b may take less time than the compression of the first chamber 51a. For example, if the compression of the first chamber 51a takes 6 seconds, the expansion of the second chamber 51b may take between 2 and 6 seconds.

When one chamber stops while the other is operating, it causes a sudden change in dialysate pressure, which subsequently affects the transmembrane pressure (TMP). This change can enhance mass transfer by altering the water flux through the membranes 12 of the blood dialyzing filter 10.

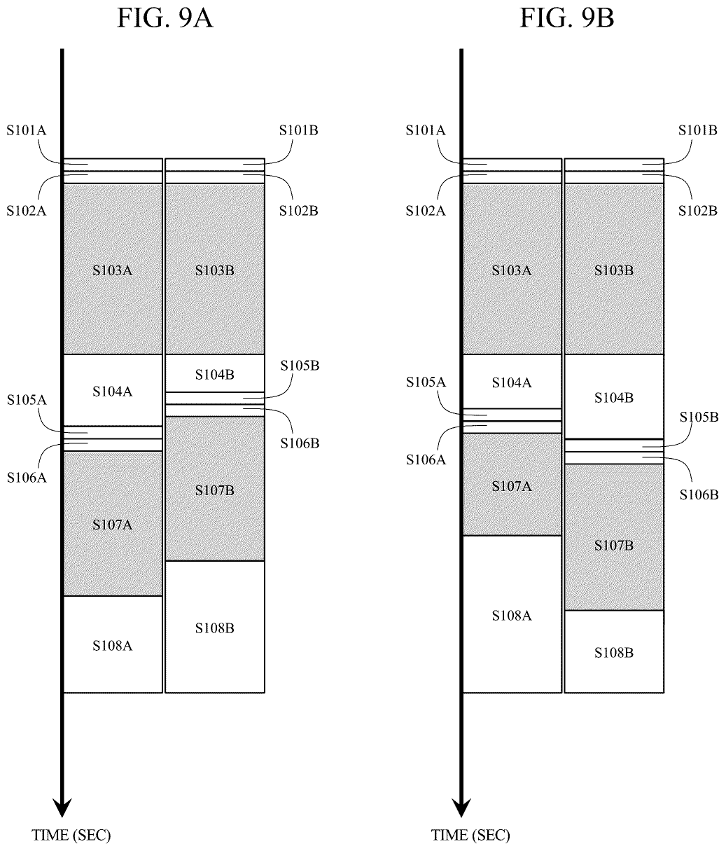

Referring to FIG. 9A, step S107B precedes step S107A, so S107B finishes earlier than S107A. To achieve this, S105B begins before S105A. Conversely, as illustrated in FIG. 9B, step S107A may begin before S107B and take less time than S107B, and consequently, step S108A to take longer than S108B.

As shown in the drawings, steps S101 to S108 can be adjusted in terms of the time taken for each step or the starting point of each step.

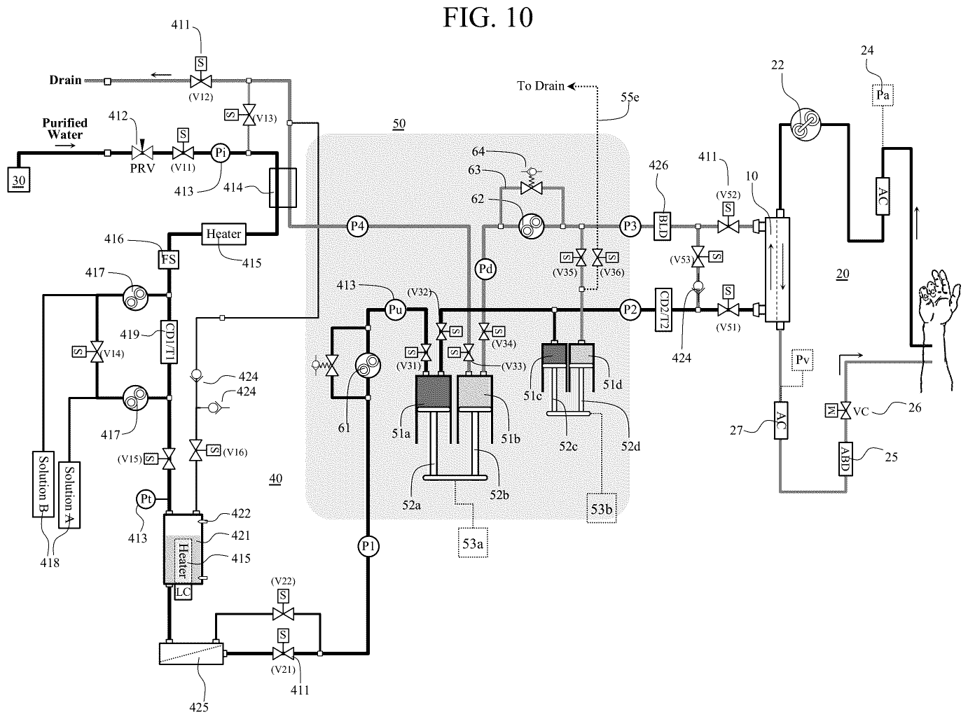

The flow control unit 50 is not limited to the structure shown in FIG. 4 and can be modified to the structure shown in FIG. 10, where additional volume chambers 51c and 51d are provided. The first volume chamber 51c is positioned downstream of the first chamber 51a, allowing it to hold dialysate flowing from the first chamber 51a. The second volume chamber 51d is placed before the second chamber 51b, enabling it to temporarily store dialysate from the blood dialyzing filter 10 before supplying it to the second chamber 51b.

Unlike the first and second chambers 51a and 51b, the first volume chamber 51c may be connected with a single tube through which dialysate flows in and out. In FIG. 10, for example, the first volume chamber 51c is connected to the dialysate inlet tube 427, which links the flow control unit 50 and the blood dialyzing filter 10, upstream of the blood dialyzing filter 10.

The second volume chamber 51d is connected to the dialysate outlet tube 428, which connects the blood dialyzing filter 10 and the flow control unit 50, downstream of the blood dialyzing filter 10. Additionally, the second volume chamber 51d may have a volume chamber bypass tube 55e, shown as a dotted line in FIG. 10, allowing dialysate to be removed from the second volume chamber 51d. Besides a flow control valve V35 installed in the tube connecting the second volume chamber 51d to the dialysate outlet tube 428, a valve V36 is also provided in the volume chamber bypass tube 55e to regulate flow through the tube 55e.

The chamber pressurizing members 52a and 52b may be assembled together to move simultaneously along a linear direction, compressing or expanding the internal spaces of chambers 51a and 51b. Similarly, the volume chamber pressurizing members 52c and 52d can be formed as a single body to move simultaneously. In this configuration, the first and second chamber pressurizing members 52a and 52b may be driven by the first chamber pressurizing member driver 53a, while the first and second volume chamber pressurizing members 52c and 52d may be driven by the second chamber pressurizing member driver 53b.

The blood dialyzing apparatus 1 is not limited to the configurations described above and may be modified in various ways. For instance, each of the chamber pressurizing members 52a to 52d can be operated by separate chamber pressurizing member drivers, allowing for the independent operation of each chamber pressurizing member. For example, the first chamber pressurizing member 52a could be operated by a first chamber pressurizing member driver 53a, the second chamber pressurizing member 52b by a second chamber pressurizing member driver 53b, and so on. Alternatively, two or more chamber pressurizing members could be operated together by the same chamber pressurizing member driver; for instance, all the chamber pressurizing members 52a to 52d could be operated by a single chamber pressurizing member driver.

When the first and second chamber pressurizing members 52a and 52b are driven by a single chamber pressurizing member driver 53a, the stroke volumes of the first and second chambers 51a and 51b can be maintained equally if the inner diameters are identical. The same applies to the volume chambers 51c and 51d.

Hereinafter, a method of operating the flow control unit 50 according to another embodiment of the present invention will be described in detail with reference to the accompanying drawings.

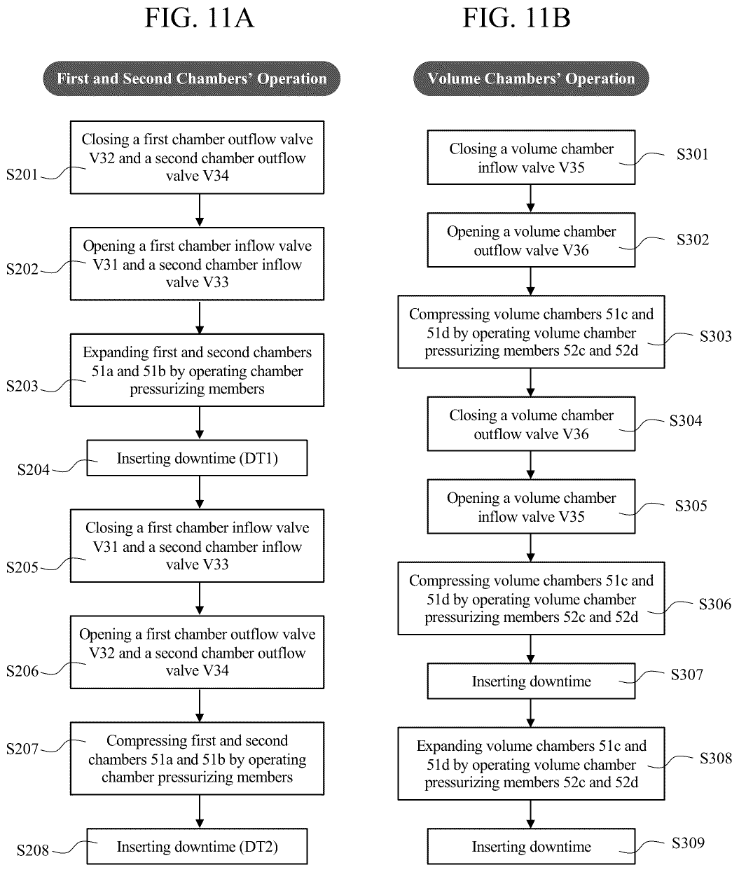

FIG. 11A is a flowchart illustrating the steps for operating the first and second chambers 51a and 51b, with their chamber pressurizing members assembled together.

The steps for operating the first and second chambers 51a and 51b in S201 through S208 are substantially similar to steps S101 to S108. However, the first and second chamber pressurizing members 52a and 52b move together as a single unit. This means that the first and second chambers 51a and 51b are compressed or expanded simultaneously. Accordingly, the sequence of blocking or unblocking tubes with valves V31 to V34 may be adjusted to allow fresh dialysate to flow toward blood dialyzing filter 10 but used dialysate to be removed from the blood dialyzing filter 10. For example, valves V32 and V34 operate together, and valves V31 and V33 operate simultaneously.

FIG. 11B is a flowchart illustrating the operational steps of the volume chambers 51c and 51d according to an embodiment of the present invention.

Steps S301 and S302 involve valve operations. Specifically, the volume chamber inflow valve V35 is closed (S301), and then the volume chamber outflow valve V36 is opened (S302).

Next, the volume chambers 51c and 51d are compressed (S303). Dialysate from the first volume chamber 51c is supplied to the dialysate stream at the dialysate inlet tube 427, and dialysate from the second volume chamber 51d is discharged through the volume chamber bypass tube 55e. Since the bypass tube 55e leads to the system drain tube, the dialysate from the second volume chamber 51d is removed from the blood dialyzing apparatus 1.

After S303, the valve status is reset to its state before S301. Namely, the volume chamber outflow valve V36 is closed, and then the volume chamber inflow valve V35 is opened (S304).

The volume chambers 51c and 51d are compressed again (S306). During this step, dialysate from the volume chambers 51c and 51d is discharged into the dialysate stream. Specifically, dialysate from the first volume chamber 51c is supplied to the dialysate inlet tube 427, and dialysate from the second volume chamber 51d is pumped to the dialysate outlet tube 428.

Since the volume chambers 51c and 51d are compressed, it may be advantageous to allow time for fluid equilibrium to be reached (S307).

Then, the volume chamber pressurizing members 52c and 52d move in the reverse direction to expand the volume chambers 51c and 51d, allowing dialysate to flow into the volume chambers (S308).

Since the volume chambers 51c and 51d are expanded, it may be beneficial to insert another downtime (S309), which can be determined based on the overall cycle time.

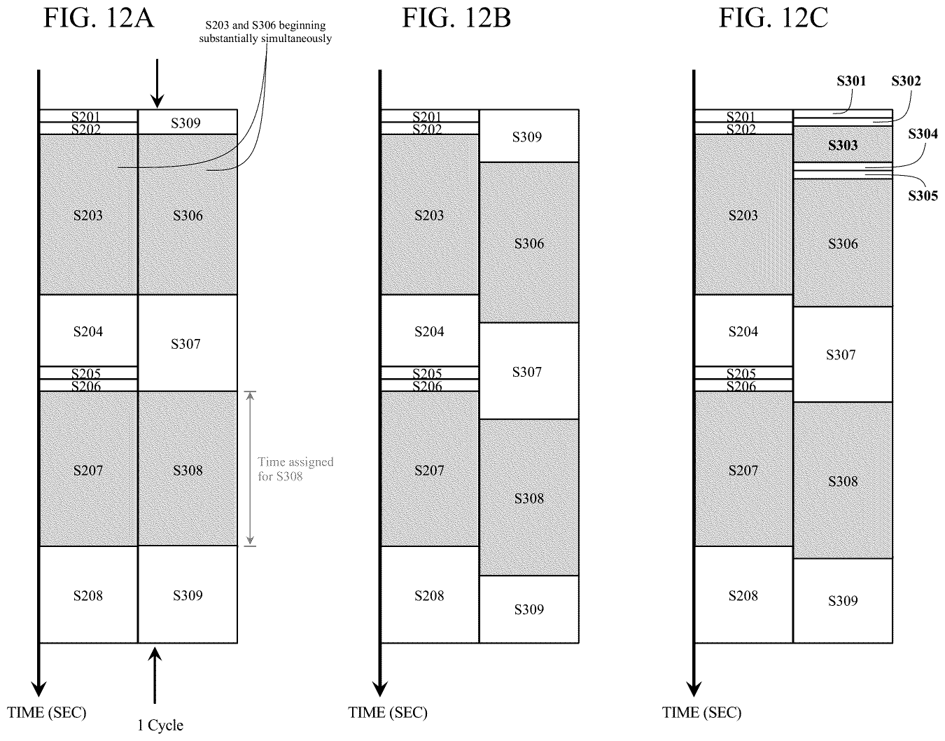

In various scenarios, the operation of the volume chambers 51c and 51d can be combined with the operation of the first and second chambers 51a and 51b, as detailed in FIGS. 12A to 12C.

In FIG. 12A, all chambers 51a to 51d may be compressed or expanded simultaneously. Steps S203 and S306 begin and end simultaneously. When step S207 starts, i.e., when the first and second chambers 51a and 51b start being compressed, step S308, where the volume chambers 51c and 51d are being expanded, also begins. Additionally, the time taken for compressing the first and second chambers 51a and 51b in step S207 is almost the same as the time taken for expanding the volume chambers 51c and 51d in step S308.

In this scenario, step S307, which allows time for fluid stabilization, takes approximately the same time as steps S204. S205, and S206. Until the operations with valves V31 to V34 are completed, no actions are taken with the volume chambers 51c and 51d.

The stroke volume (SV) of the first volume chamber 51c (SV51c) may be smaller than the SV of the first chamber 51a (SV51a). Similarly, the SV of the second volume chamber 51d (SV51d) may be smaller than the SV of the second chamber 5ib (SV51b). As shown in FIG. 12A, while compressing the first and second chambers 51a and 51b, the volume chambers 51c and 51d are expanded, allowing dialysate in the first chamber 51a to be pumped into the volume chambers 51c and 51d. Therefore, the sum of SV51c and SV51d may be similar to SV51a. Assuming SV51c and SV51d are substantially equal to each other, they may each have a value approximately half of SV51a. In addition, while expanding the first and second chambers 51a and 51b, the volume chambers 51c and 51d are compressed, allowing dialysate of the volume chambers 51c and 51d to be supplied to the second chamber 51b. Therefore, the sum of SV51c and SV51d may be similar to SV51b. The SV value ranges described herein are exemplary and can be modified as needed.

The operation of the volume chambers 51c and 51d in combination with the first and second chambers 51a and 51b can also follow the sequences shown in FIG. 12B. Step S203 precedes S306, and S207 begins before step S308, with the time for S203 and S306 remaining similar. However, as shown in FIG. 12C, the first compression of the volume chambers 51c and 51d begins before step S203, while the second compression of the volume chambers happens after step S203 has started.

Since the first and second chambers 51a and 51b, as well as the volume chambers 51c and 51d, repeat an operational cycle, it is necessary to assign approximately the same amount of time for the cycle for both sets of chambers. As mentioned earlier, in an embodiment, the cycle time may be set between 4.2 and 56 seconds, more preferably between 6.4 and 33 seconds. The operations of the first and second chambers 51a and 51b, along with the volume chambers 51c and 51d, may be synchronized to ensure that each cycle lasts the same duration.

The flow control unit 50 according to an embodiment of the present invention is not limited to the structures shown in the drawings, and may be modified into other configurations.

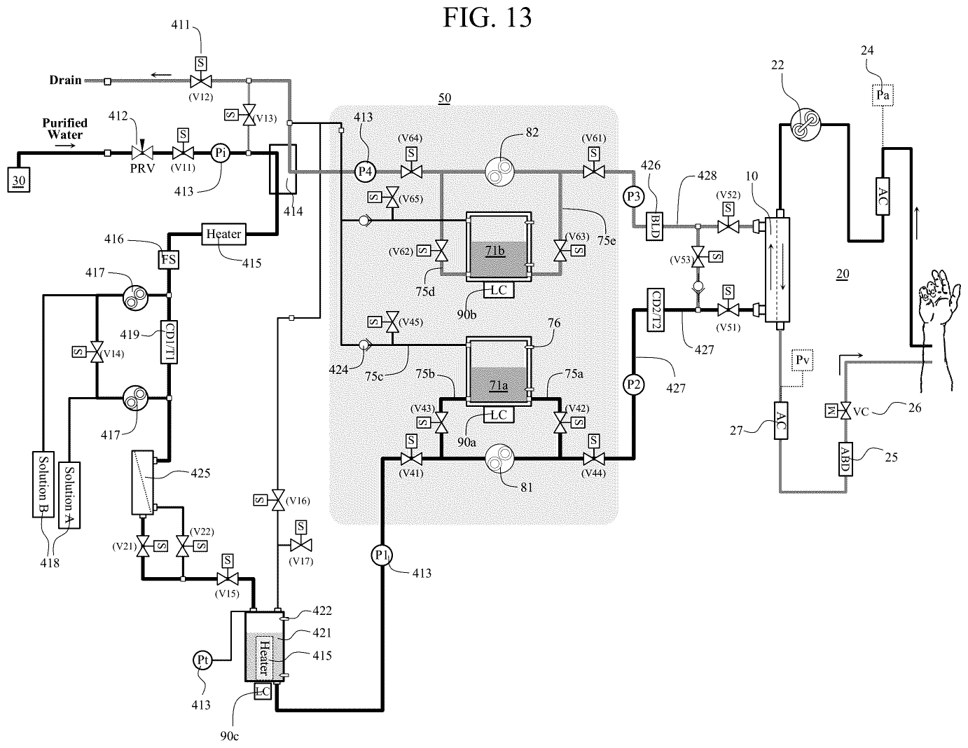

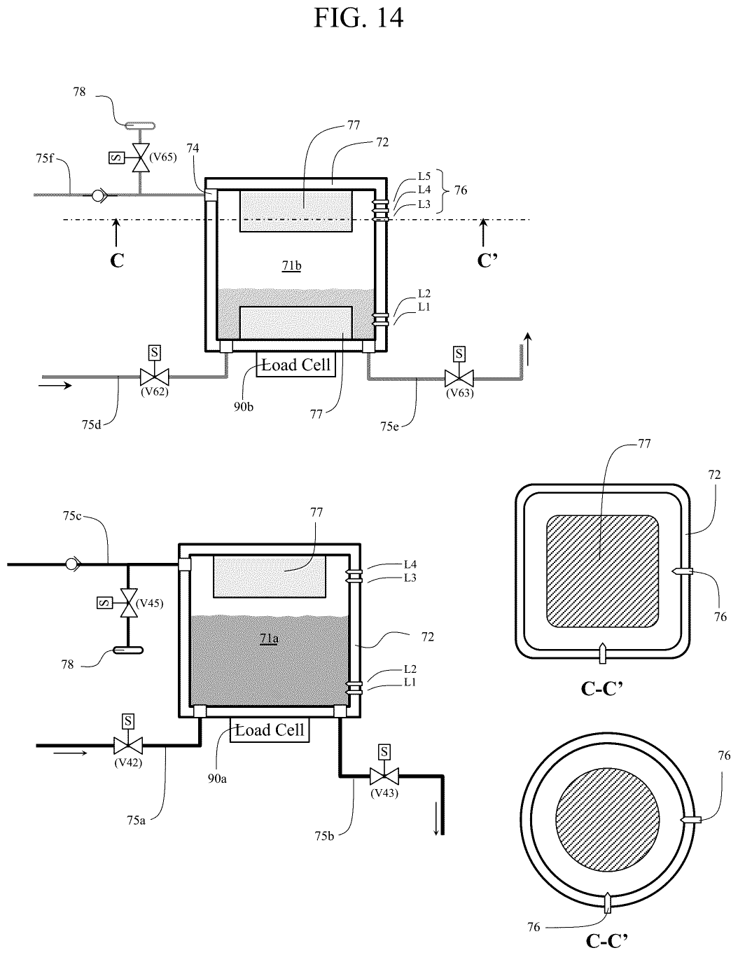

FIG. 13 is a view illustrating the flow control unit 50 of the blood dialyzing apparatus 1 according to another embodiment of the present invention. FIG. 14 is an enlarged view illustrating first and second fluid containers 71a and 71b of the flow control unit 50.

With reference to FIG. 13, the flow control unit 50 is configured to include first and second fluid containers 71a and 71b, each having an internal space to accommodate fluid, and a load cell 90 to measure the weight of the fluid containers. The first fluid container 71a holds dialysate upstream of the blood dialyzing filter 10, while the second fluid container 71b contains dialysate that has passed through the blood dialyzing filter 10. The load cell 90 may further include a first load cell 90a to measure the weight of the first fluid container 71a and a second load cell 90b to measure the weight of the second fluid container 71b. An additional load cell 90c may be installed to measure the weight of the buffer tank 421.

Each fluid container 71a and 71b has multiple fluid ports 74, with flow tubes 75 connected to these ports, enabling dialysate to flow through the containers. For example, the first fluid container 71a is connected to a first container inflow tube 75a for dialysate to enter, a first container outflow tube 75b for dialysate to exit, and a first container vent tube 75c for dialysate or air flow. Similarly, the second fluid container 71b is connected to a second container inflow tube 75d, a second container outflow tube 75e, and a second container vent tube 75f, allowing dialysate and air to flow in and out.

To ensure smooth dialysate flow through the first and second fluid containers 71a and 71b, the flow control unit 50 may be equipped with fluid pumps. In FIG. 13, a first fluid pump 81 near the first fluid container 71a transfers dialysate through the first fluid container 71a, and a second fluid pump 82 transfers dialysate through the second fluid container 71b. In other words, the operation of the first fluid pump 81 causes dialysate to flow into and out of the first fluid container 71a. Similarly, the operation of the second fluid pump 82 produces dialysate to flow into and out of the second fluid container 71b.

Any type of volume displacement pump can be used for the fluid pumps 81 and 82, such as gear pumps, peristaltic pumps, lobe pumps, rotary piston pumps, piston pumps, and others.

As the first fluid container 71a is filled or emptied, the first container vent tube 75c allows air to flow, ensuring stable pressure conditions. In addition, the fluid pumps 81 and 82 may include a bypass tube connecting the inlet and outlet sides of the pump, with a relief valve installed on the bypass tube (not shown in the drawing).

Flow control valves 411 are installed to regulate the flow passages through the tubes connected to the fluid containers. For example, a first container upstream valve V41, a first container inflow valve V42, a first container outflow valve V43, a first container downstream valve V44, and a first container vent valve V45 are installed in the tubes connected, directly or indirectly, to the first fluid container 71a. Similarly, a second container upstream valve V61, a second container inflow valve V62, a second container outflow valve V63, a second container downstream valve V64, and a second container vent valve V65 are installed in the tubes connected to the second fluid container 71b. These valves control the flow passages through the tubes, directing the dialysate flow in collaboration with the fluid pumps 81 and 82. Exemplary flow control valves include solenoid valves, which are controlled by electric signals, metering valves, clamp valves, and the like. According to an embodiment of the present invention, one-way check valves may further be installed in the vent tubes 75c and 75f as shown in FIG. 13.

The housing 72 of the fluid containers may be made of a substantially inflexible material such as plastic, metal, or acrylic, with a cross-sectional shape of a triangle, circle, or rectangle. To detect the fluid level inside the container, level sensors 76 may be installed within the container housing 72. These include a bottom limit level sensor 76a and an upper limit level sensor 76b. For example, L1 in FIG. 14 corresponds to the bottom limit level sensor 76a, while L4 and L5 correspond to the upper limit level sensors 76b. In the drawing, the first fluid container 71a includes four level sensors 76 (L1 to L4), and the second fluid container 71b includes five level sensors 76 (L1 to L5). The number and placement of level sensors can be modified as needed to ensure the safe operation of the flow control unit 50. Multiple level sensors 76 are provided vertically, and two or more level sensors may be installed around the circumference of the container to detect the same level.

As dialysate passes through containers 71a and 71b, a stroke volume (SV) and a flow rate for a container can be defined as the volume of dialysate that fills and empties the container over a preset time. For example, if the first fluid container 71a is filled with 500 mL of dialysate in 24 seconds and then emptied the same amount in 36 seconds, the SV of the first fluid container 71a is 500 mL, while the flow rate is 500 mLmin (milliliter per minute).

The SV of the first fluid container 71a (SV71a) may be substantially the same as that of the second fluid container 71b (SV71b). That is, the second fluid container 71b may be filled with roughly the same amount of dialysate discharged from the first fluid container 71a. However, according to an embodiment of the present invention, SV71b may be larger than SV71a. For example, when the first fluid container 71a supplies 500 mLmin of dialysate to the blood dialyzing filter 10 and the second fluid container 71b draws 510 mLmin from it, the difference in SV between the first and second fluid containers 71a and 71b, i.e., SV71a-SV71b, represents the net fluid removal from the patient, termed net ultrafiltration (UF), which is −10 mL/min in the example. Here, a negative net UF (SV71a−SV71b<0) indicates fluid loss from the patient, whereas a positive net UF indicates fluid addition to the patient's bloodstream.

The SV71a and SV71b, according to an embodiment of the present invention, may be set to a value ranging from 100 mL to 800 mL, preferably 120 mL to 680 mL, and more preferably 140 mL to 280 mL. Additionally, the SV71b value may be larger than SV71a by 0 to 20% of SV71a, more preferably by 0 to 10% of SV71a. The cycle time can be determined by considering the required dialysate flow amount per minute and the SV71a and SV71b values.

The net UF can be achieved by regulating the speed of pumps 81 and 82, adjusting the pump operating time, or controlling the valve operation time, such as how long a valve remains open. In an embodiment, the pump operation (including pump speed and running time) and the valve operation (including valve ON and OFF intervals) can be controlled by the level sensors 76, by the load cell values, or by a combination thereof.

Air flows through the vent tubes 75c and 75f, each equipped with flow control valves V45 and V65 on tubes connected to the vent tubes 75c and 75f, as shown in FIG. 14. An air filter 78 may be installed at the end of these tubes to prevent harmful substances from entering the containers when air flows in. Various types of air filters can be used for this purpose.

Protruding bodies 77 may also be placed inside the fluid containers 71a and 71b. These protruding bodies 77 serve to decrease the cross-sectional area at a certain level inside the fluid container housing 72. In a preferred embodiment, the protruding bodies 77 are positioned at the levels detected by the bottom limit level sensor 76a and the upper limit level sensor 76b. Load cells 90a and 90b are positioned at the bottom of the first and second fluid containers 71a and 71b, respectively, to measure their weights.

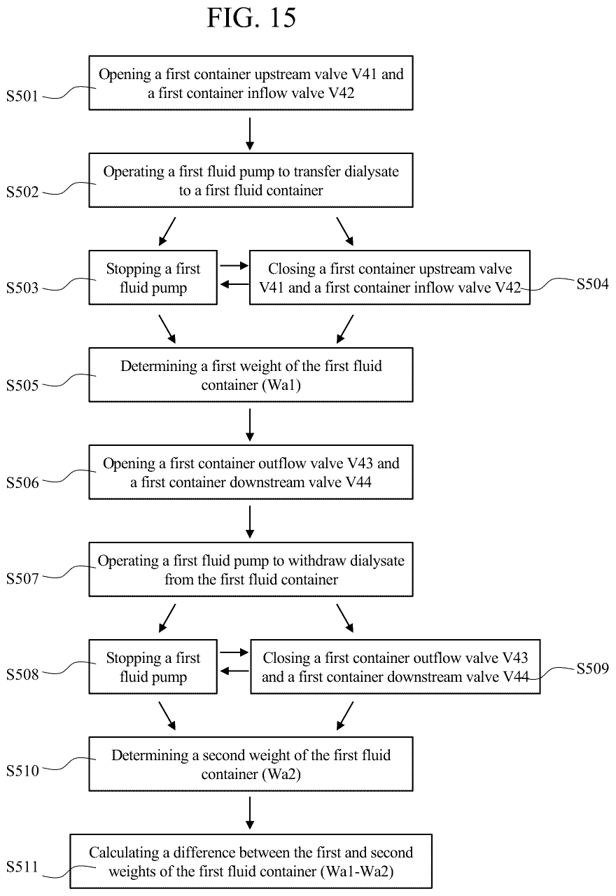

Hereinafter, an operation of the flow control unit 50 according to an embodiment of the present invention will be described in detail with reference to the accompanying drawings. FIG. 15 is a flowchart illustrating the steps of operating the first fluid container 71a.

### (1) S501

Step S501 involves valve operation. Valves V41 and V42 are opened, as valves V41 through V44 were closed at the end of the previous cycle.

### (2) S502

Upon opening the valves, the first fluid pump 81 begins operating, supplying dialysate to the first fluid container 71a. Therefore, step S502 is referred to as the ‘dialysate filling’ phase. As the first fluid container 71a is filled with dialysate, air inside the container is vented out through the first container vent tube 75c because the first container vent valve V45 remains open.

During this step, the fluid level inside the first fluid container 71a rises, and the weight of the first fluid container 71a increases, which is detected by the first load cell 90a.

### (3) S503 and S504

Steps S503 and S504 are implemented to prevent the first fluid container 71a from being overfilled with dialysate. When the first fluid container 71a receives a predetermined amount of dialysate, it is necessary to stop further flow into the container. One method is to stop the first fluid pump 81, thereby halting the supply of dialysate to the first fluid container 71a (S503).

Determining when the first fluid container 71a is filled with the preset amount of dialysate can be achieved through various methods, such as:
1. When the fluid level inside the first fluid container 71a reaches a predetermined upper limit level sensor 76b.
2. When the first load cell 90a detects a preset weight.
3. When the first fluid pump 81 runs for a predetermined amount of time.
4. A combination of the above methods.

For example, the first fluid pump 81 may stop when the upper limit level sensor 76b detects dialysate, or when the first load cell 90a measures a weight increase by a predetermined amount. Alternatively, the pump may stop after running for a specific period. The method of operating the flow control unit 50 is not limited to these methods and may include other ways to precisely determine when to halt filling.

The method of operating the flow control unit 50 may be configured to include a step where the first container inflow valve V42 and/or the first container upstream valve V41 may be closed (S504), either instead of or in addition to stopping the first fluid pump 81. When these valves are closed, no dialysate can enter the first fluid container 71a.

The operation steps of the flow control unit 50 can also be modified such that step S504 (i.e., closing valves) precedes S503 (i.e., stopping a pump), or steps S503 and S504 are performed simultaneously, as shown in FIG. 15.

### (4) S505

When the pumps 81 and 82 are stopped and valves V41 to V44 are closed, the flow control unit 50 measures the weight of the first fluid container 71a using the first load cell 90a. This measurement determines and records the first weight (Wa1) of the first fluid container 71a.

### (5) S506

The description provided for step S501 can be similarly applied to step S506.

### (6) S507

The description for step S502 can be similarly applied to step S507, except that the filling or emptying of dialysate is reversed. In step S507, the first fluid pump 81 runs to discharge the dialysate toward the blood dialyzing filter 10. Thus, S507 is referred to as the ‘dialysate supplying’ phase.

As dialysate is removed in step S507, the fluid level inside the first fluid container 71a decreases, and the weight of the first fluid container 71a, as detected by the first load cell 90a, also decreases.

### (7) S508 and S509

Substantially the same description used for steps S503 and S504 can be applied to S508 and S509.

Since dialysate is removed from the first fluid container 71a during step S507, the pump may be stopped (S508) or the valves may be closed (S509) when the fluid level inside the first fluid container 71a reaches the predetermined bottom limit level sensor 76a, or when the weight decreases by a predetermined amount. Additionally, the pump stop, or valve closure can be performed after a predetermined period of time, such as 30 seconds.

### (8) S510 and S511

When the fluid pump 81 is stopped and valves V43 and V44 are closed, the flow control unit 50 measures the weight of the first fluid container 71a. This measurement determines the second weight (Wa2) of the first fluid container 71a. The difference between Wa1 and Wa2 is then calculated to determine how much dialysate has been supplied to the blood dialyzing filter 10 from the first fluid container 71a.

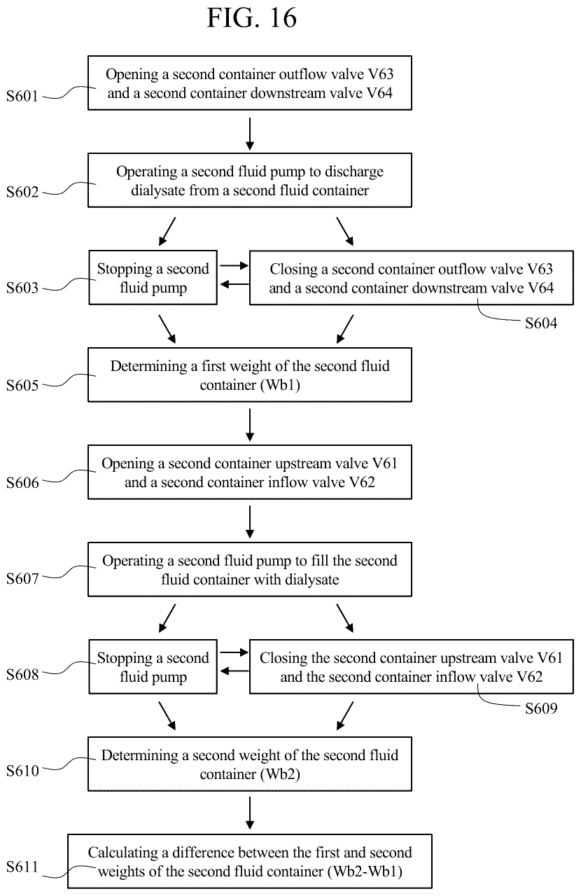

FIG. 16 is a flowchart illustrating the steps of operating the second fluid container 71b. Steps S601 through S611 shown in FIG. 16 are substantially similar to steps S501 through S511 shown in FIG. 15. For example, in terms of function. V41 corresponds to V61, V42 to V62, V43 to V63, V44 to V64, and the first fluid pump 81 corresponds to the second fluid pump 82. Additionally, when the first fluid container 71a is filled with dialysate by the first fluid pump 81 (S502), the second fluid container 71b may be emptied by the second fluid pump 82 (S602), and vice versa.

When the weight of the first fluid container 71a decreases (S507), the weight of the second fluid container 71b preferably increases (S607). In other words, when dialysate is supplied from the first fluid container 71a to the blood dialyzing filter 10 by the first fluid pump 81, dialysate may simultaneously be supplied from the blood dialyzing filter 10 to the second fluid container 71b by the second fluid pump 82.

The steps shown in FIG. 15 and FIG. 16 represent a complete cycle of operation for the first fluid container 71a and the second fluid container 71b, respectively. The first and second fluid containers repeat this cycle during the operation of the blood dialyzing apparatus 1.

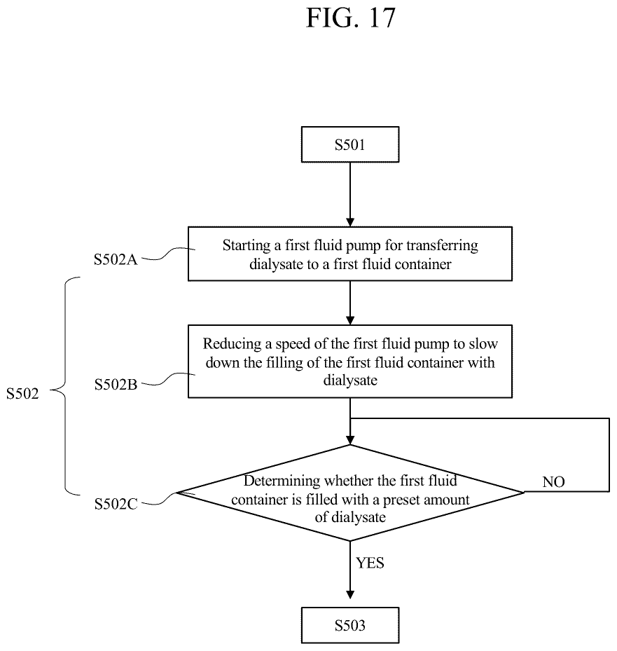

The fluid pumps 81 and 82 operate to transfer dialysate to or from the respective fluid containers during steps S502, S507, S602, and S607. Referring to FIG. 17, step S502 may further include the following steps:

Starting the first fluid pump 81 to begin transferring dialysate to the first fluid container (S502A).

Reducing the speed of the first fluid pump 81 to slow down the filling of dialysate into the first fluid container (S502B).

Determining whether the first fluid container is filled with the predetermined amount of dialysate (S502C).

These sub-steps can also be applied to steps S507, S602, and S607 because they also involve pump operation.

The first fluid container 71a needs to be filled with a predetermined amount of dialysate. Accurately filling the container with the precise amount of dialysate is important for regulating the patient's hydration level. To aid this process, it may be necessary to adjust the pump speed, either increasing or decreasing it, to minimize any error in the dialysate volume inside the first fluid container 71a.

The dialysate volume supplied to the blood dialyzing filter 10 by the first fluid pump 81 is determined at step S511 (i.e., Wa1−Wa2, corresponding to SV71a per cycle). Similarly, the dialysate volume pumped into the second fluid container 71b from the blood dialyzing filter 10 by the second fluid pump 82 is determined at step S611 (i.e., Wb2−Wb1, corresponding to SV71b per cycle).

The method of operating the flow control unit 50, according to an embodiment of the present invention, may further include adjusting the pump speed and/or adjusting the pump operating time for the next cycle based on the SV71a or SV71b values obtained in the preceding cycle. For example, if the SV71a value is smaller than a preset value, the operating speed or time for the first fluid pump 81 may be increased in the following cycle or cycles.

Per each cycle, the SV71a and SV71b values may range from 10 to 800 grams per cycle, more preferably 200 to 600 grams per cycle. As mentioned above, SV71b per cycle may be maintained equally to or be set to greater values than SV71a per cycle.

Additionally, the cycle time for the operation of the first and second fluid containers 71a and 71b may be configured to take between 10 and 600 seconds, more preferably between 20 and 120 seconds. The cycle time needs to be determined considering the stroke volumes of the first and second fluid containers 71a and 71b.

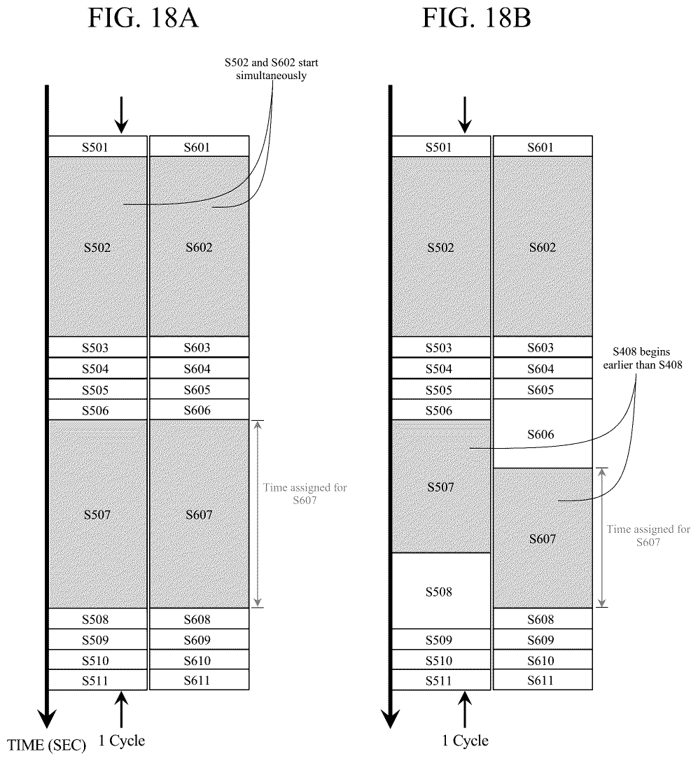

FIGS. 18A and 18B are schematic views illustrating the operational sequences of the first fluid container 71a and the second fluid container 71b according to an embodiment of the present invention.

As shown in FIG. 18A, the operational steps for the first and second fluid containers 71a and 71b can be synchronized, meaning that the steps for both containers begin and end at approximately the same time. Additionally, the time assigned to step S502 may be equal to that assigned to step S507, meaning it takes roughly the same time to fill and then empty the first fluid container 71a.

However, the method of operating the flow control unit 50 is not limited to this sequence and can be modified so that the time for step S502 is shorter than the time for step S507. Specifically, the first fluid container 71a can be filled with dialysate in less time than it takes to empty it. In other words, the ‘dialysate supplying’ phase lasts longer than the ‘dialysate filling’ phase. This approach is particularly advantageous because a longer supply phase for dialysate to the blood dialyzing filter 10 enhances the removal efficiency of uremic marker molecules.

In contrast to FIG. 18A, in FIG. 18B, step S507 begins before step S607. This means that dialysate from the first fluid container 71a is pumped into the blood dialyzing filter 10 while no dialysate is supplied to the second fluid container 71b, leading to a net fluid supply to the bloodstream, known as backfiltration. However, even though the first fluid pump 81 stops at the end of S507, the second fluid pump 82 continues to draw dialysate from the blood dialyzing filter 10, resulting in a net fluid removal from the patient's blood, known as ultrafiltration.

It is apparent to those skilled in the art that the operational sequence can be modified so that step S607 precedes step S507. The same modification can also be applied to steps S502 and S602 without any restrictions. Therefore, by alternating the sequences of steps S507 and S607, the blood dialyzing apparatus 1 can increase mass transfer between blood and dialysate in the blood dialyzing filter 10, providing improved efficiency for the patient.

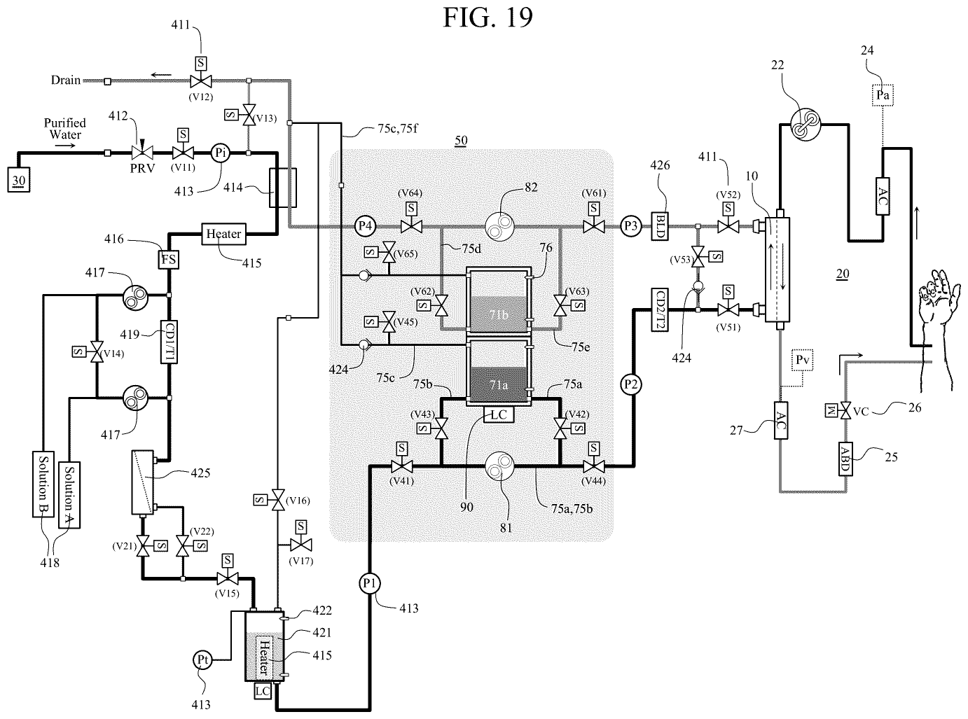

FIG. 19 is a view illustrating the flow control unit 50 of the blood dialyzing apparatus 1 according to another embodiment of the present invention.

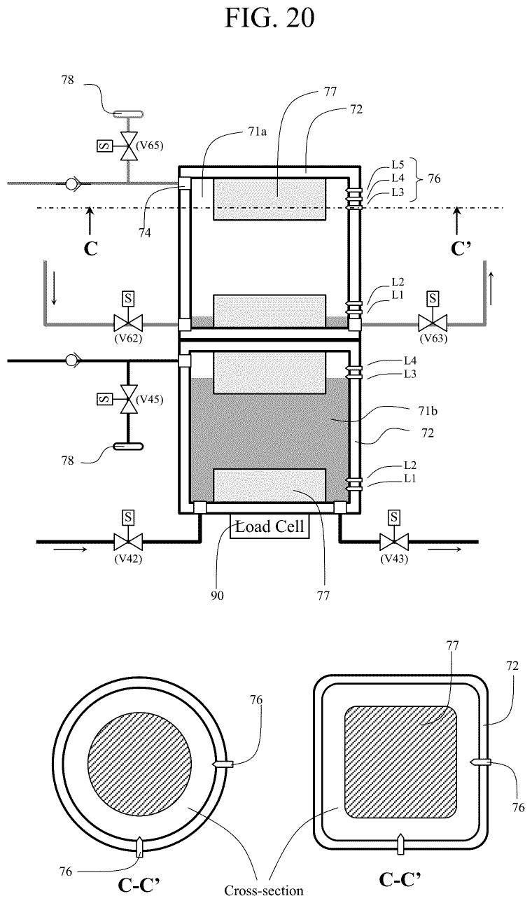

Referring to FIG. 19, the first and second fluid containers 71a and 71b may be stacked along a vertical direction. In order to increase the stability upon combining the two containers, the first and second fluid containers 71a and 71b may have the same cross-sectional shape with each other, such as circular, triangular, or rectangular. Unlike the first fluid container 71a, the second fluid container 71b may have the fluid ports 74 at a side surface thereof, as shown in FIG. 20.

The load cell 90 may be positioned at a bottom of the ‘combined’ first and second fluid containers 71a and 71b to measure their weight.

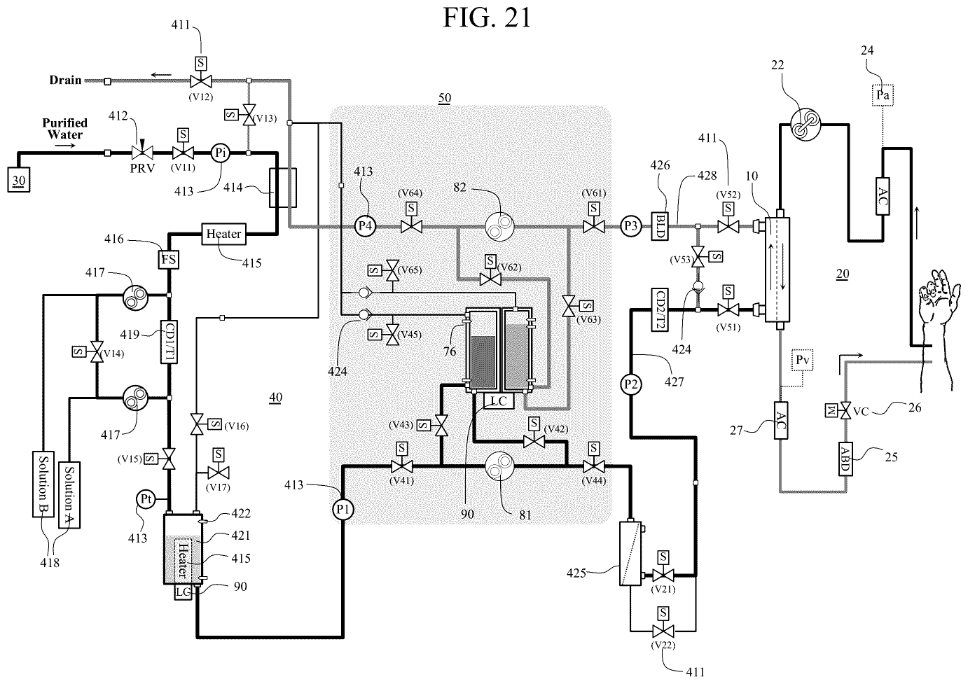

Here, the arrangement of the first and second fluid containers 71a and 71b is not limited to a vertical stack; they can also be configured horizontally, placed next to each other, as shown in FIG. 21.

Because the first and second containers are assembled, the operation of the combined container can be configured by integrating the operations of each individual container, except that the load cell 90 measures the weight of the assembled containers.

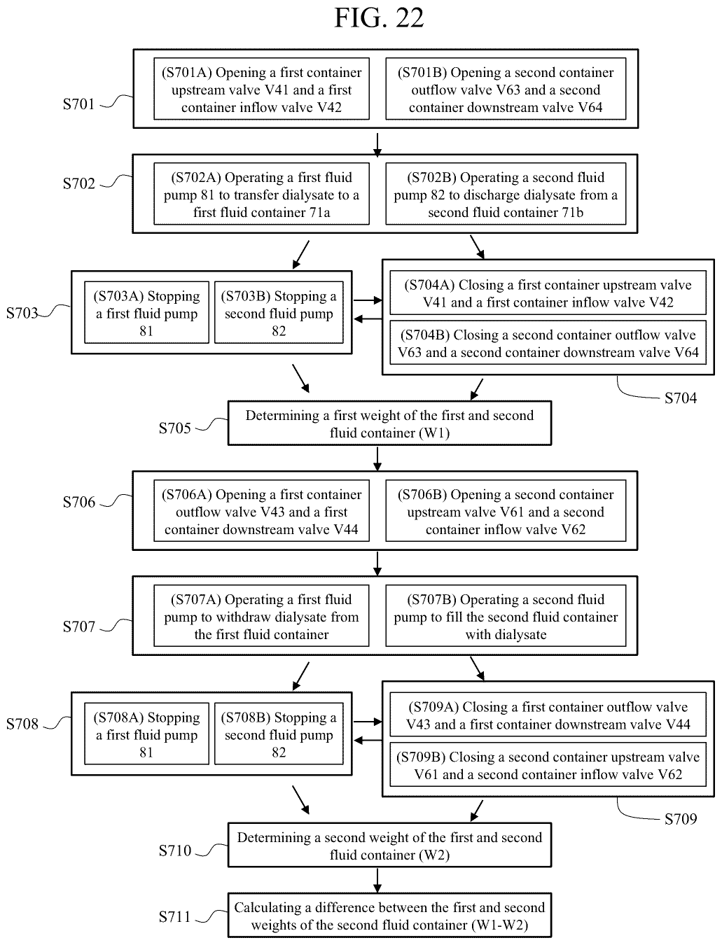

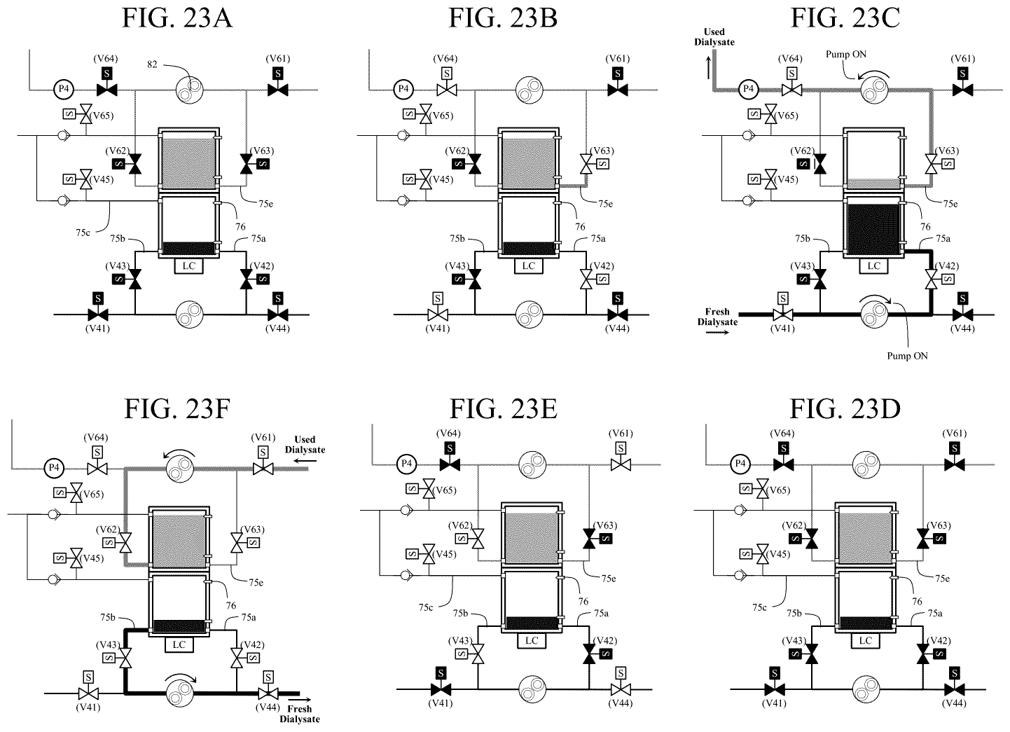

FIG. 22 is a flowchart illustrating the steps of operating the flow control unit 50. Steps S701 through S711 constitute a full cycle of the operation. FIGS. 23A to 23F provide schematic circuit diagrams of the flow control unit 50 at each stage of the operation.

### (1) S701

Step S701 involves valve operation. Valves V41 and V42 for the first fluid container 71a and valves V63 and V64 for the second fluid container 71b are opened. This is necessary because valves V41 through V44 and valves V61 through V64 are closed at the end of the previous cycle. Step S701 may further be divided into S701A for opening valves V41 and V42 and S701B for opening valves V63 and V64. This step is repoesented in FIGS. 23A and 23B.

Since S701A and S701B are substantially similar to S501 and S601, respectively, the descriptions used for S501 and S601 can be applied to S701.

### (2) S702

Upon opening the valves, the fluid pumps 81 and 82 begin operating at step S702. This step, represented by FIGS. 23B to 23C, further includes steps S702A and S702B, which involve the operation of the first fluid pump 81 and the second fluid pump 82, respectively.

As illustrated in FIG. 23C, the first fluid pump 81 supplies dialysate to the first fluid container 71a, while the second fluid pump 82 discharges dialysate from the second fluid container 71b. In the drawing, a thick line indicates that dialysate is flowing through the tube (i.e., the valve is open), while a thin line indicates no dialysate flow. Thus. S702 is referred to as the ‘dialysate filling’ phase.

When the first fluid container 71a is filled with dialysate, air inside the container is vented out through the first container vent tube 75c because the first container vent valve V45 is open. Conversely, air flows into the second fluid container 71b as it empties, due to the second container vent valve V65 being open.

Since S702A and S702B are substantially similar to S502 and S602, respectively, the descriptions used for S502 and S602 can be applied to S702.

### (3) S703 and S704

Substantially the same descriptions used for steps S503 and S504, as well as for steps S603 and S604, can be applied to steps S703 and S704. Step S704 is depicted in FIGS. 23C to 23D.

### (4) S705

When the pumps 81 and 82 are stopped and the valves V41 to V44 and V61 to V64 are closed, the flow control unit 50 measures the weight of the first and second fluid containers 71a and 71b. This measurement determines and records the first weight (W1) of the fluid containers 71a and 71b.

### (5) S706

Substantially the same description for S701 can be applied to S706. The step S706 is represented by FIG. 23D to 23E.

### (6) S707

The description used for step S702 can be similarly applied to step S707, except that the filling and emptying of dialysate are reversed compared to S702. In step S707, the first fluid pump 81 supplies dialysate from the first fluid container 71a to the blood dialyzing filter 10, and the second fluid pump 82 transfers dialysate from the blood dialyzing filter 10 to the second fluid container 71b. Thus, S707 may be referred to as the ‘dialysate supplying’ phase. Step S707 is represented in FIGS. 23E to 23F.

### (7) S708 and S709

The description used for S703 and S704 may be applied similarly to S708 and S709. FIG. 23F to 23A represents the step S709.

### (8) S710 and S711

Similar to step S705, when the fluid pumps 81 and 82 are stopped and valves V41 to V44 and V61 to V64 are all closed, the flow control unit 50 measures the weight of the first and second fluid containers 71a and 71b again. This measurement determines the second weight (W2) of the fluid containers 71a and 71b.

Next, the difference between W1 and W2, i.e., W1−W2, is calculated (S711) to determine how much fluid has been removed from the blood compartment of the blood dialyzing filter 10, termed the net UF rate.

The value W1−W2 (termed ‘SV71ab’) may be smaller than the value of SV71a or SV71b. For example, the SV71a or SV71b value ranges between 10 grams and 800 grams per cycle (with a cycle duration of one minute), and more preferably between 150 grams and 600 grams per minute, the value SV71ab may range between 0 and 50 grams, and more desirably between 0 and 30 grams. That is, in a structure where the first and second fluid containers 71a and 71b are combined, the variation in weight values, i.e., W1-W2, detected by the load cell 90 is relatively smaller than the value Wa1−Wa2 or Wb1−Wb2.

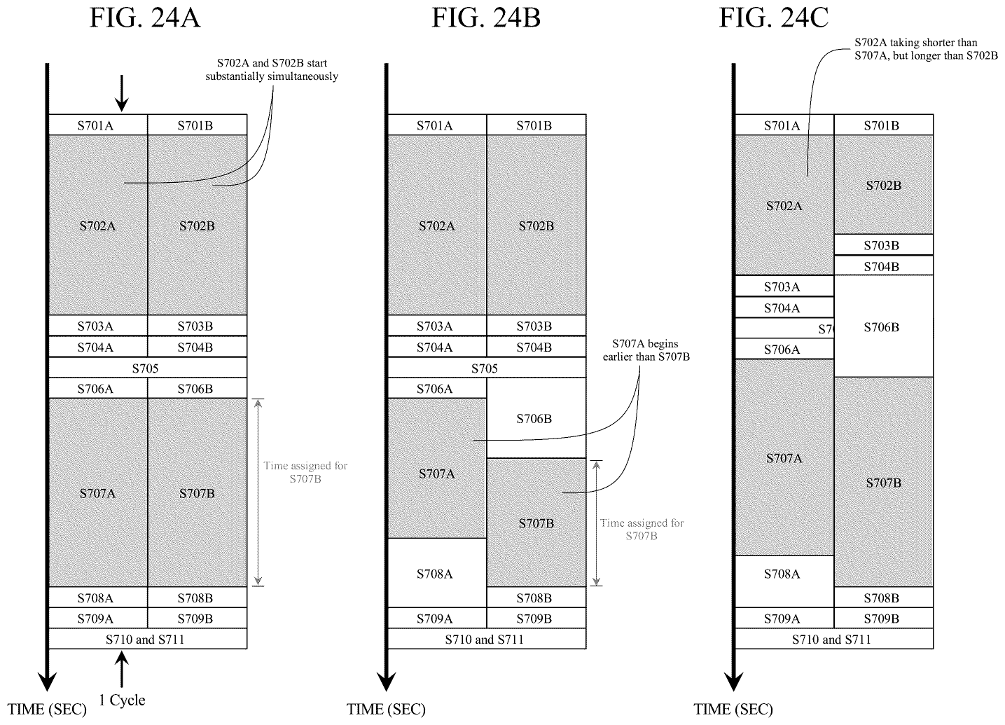

Various operational sequences of the first and second fluid containers 71a and 71b are also provided in FIGS. 24A to 24C, which are substantially similar to the operational sequences shown in FIGS. 18A and 18B.

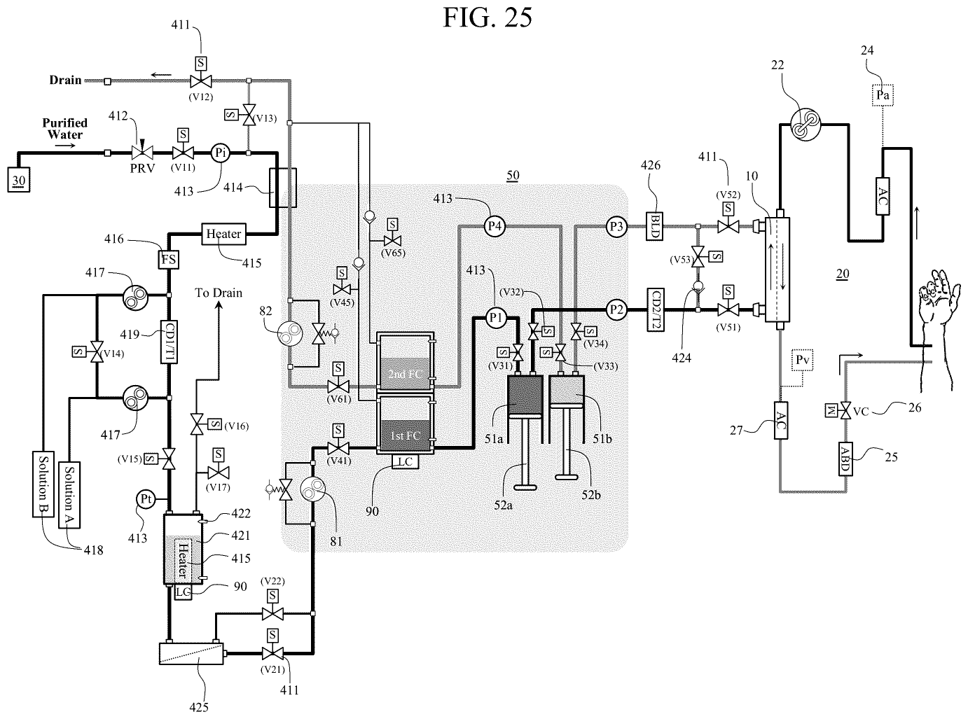

The flow control unit 50 according to an embodiment of the present invention is not limited to the previously described structures. FIG. 25 illustrates the flow control unit 50 according to another embodiment of the present invention.

This embodiment of the flow control unit 50 includes both chambers and containers: the first and second chambers 51a and 51b (as described in FIGS. 4 and 5) and the first and second fluid containers 71a and 71b (as described in FIGS. 19 and 20). Therefore, while FIG. 25 is largely self-explanatory, the descriptions provided for FIGS. 4 and 5, and FIGS. 19 and 20, can be similarly applied to the flow control unit 50 shown in FIG. 25.

Hereinafter, a method of calibrating the load cell 90 according to an embodiment of the present invention will be described in greater detail with reference to the accompanying drawings.

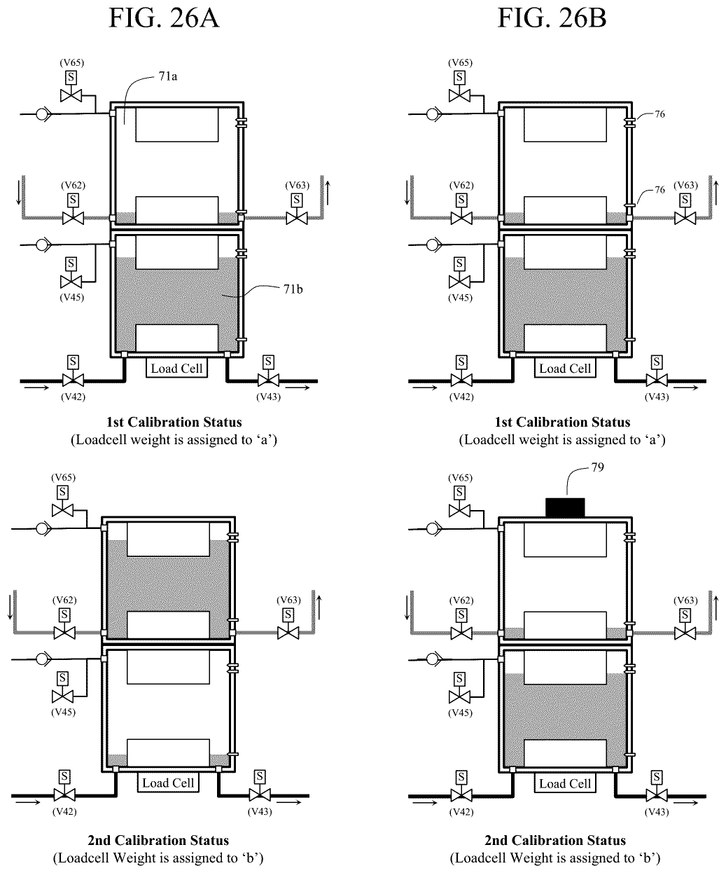

FIGS. 26A and 26B are schematic diagrams for calibrating the load cell 90 according to an embodiment of the present invention. To calibrate the load cell 90, two known weight values may be applied. For instance, let the first weight value be referred to as ‘a’ and the second weight value as ‘b’, as shown in FIG. 26A. The state ‘a’ may be defined when the first fluid container 71a is at the upper limit level sensor 76b and the second fluid container 71b is at the bottom limit level sensor 76a.

Conversely, the state ‘b’ may be defined when the first fluid container 71a is at the bottom limit level sensor 76a and the second fluid container 71b is at the upper limit level sensor 76b. Different weight values can be assigned to ‘a’ and ‘b’ due to the varying fluid weights detected by these sensors when the bottom and upper limit level sensors are established.

The flow control unit 50 according to one embodiment of the present invention is not limited to the aforementioned calibration method. For example, as shown in FIG. 26B, it may also be calibrated using a weight 79 with a known mass.

The calibration method shown in FIGS. 26A and 26B is intended to be exemplary. The load cell 90 of the flow control unit 50, according to an embodiment of the present invention, can be calibrated using various methods beyond those illustrated in the drawings. Similarly, in the case of calibration using fluid levels as shown in FIG. 26A, different fluid levels can be used to define the states ‘a’ and ‘b’.

The blood dialyzing apparatus 1 uses the fluid chambers 51a to 51d, or the first and second fluid containers 71a and 71b, as the means of transferring dialysate to the blood dialyzing filter 10 to conduct the blood dialyzing treatment using the blood dialyzing apparatus 1. However, the blood dialyzing apparatus 1 according to an embodiment of the present invention is not limited thereto, and obviously can be modified into another structure.

Provided is the blood dialyzing apparatus according to an embodiment of the present invention, in which multiple fluid chambers are compressed and expanded to transfer dialysate. The multiple chambers ensure dialysate flow rates upstream and downstream of the blood dialyzing filter to be regulated independently or precisely. Neither a separate ultrafiltration pump, nor a separate balancing chamber are required. Therefore, the entire system can be sufficiently miniaturized and light-weighted, and easy to be installed while reducing the cost for blood dialyzing treatment. The blood dialyzing apparatus will eventually be an optimal alternative for the blood dialyzing treatment in a place out of hospitals.

The above descriptions are provided for illustrative purposes of the technical concepts of the present invention, and it is obvious that a person having ordinary skill in the art may variously modify and change without departing from the natural characteristics of the present invention. Therefore, the exemplary embodiments disclosed in the present invention are provided for the sake of descriptions, not limiting the technical concepts of the present invention, and it should be understood that such exemplary embodiments are not intended to limit the scope of the technical concepts of the present invention. The protection scope of the present invention should be understood by the claims below, and all the technical concepts within the equivalent scopes should be interpreted to be within the scope of the right of the present invention.

What is claimed is:

1. A blood dialyzing apparatus comprising:
a blood dialyzing filter;
a blood flowing unit allowing blood to flow through the blood dialyzing filter, and
a dialysate flowing unit allowing dialysate to be prepared and transferred through the blood dialyzing filter, the dialysate flowing unit including a flow control unit for regulating the dialysate flow through the blood dialyzing filter, wherein
the flow control unit comprising:
first and second chambers each having an internal space and connected with an inflow tube and an outflow tube, wherein a fluid is provided to the chamber through the inflow tube and the fluid of the chamber is discharged through the outflow tube;
a chamber pressurizing member disposed inside each of the first and second chambers and compressing or expanding the internal space thereof,
a chamber pressurizing member driver operating the chamber pressurizing members, and
flow control valves installed in the inflow and outflow tubes of the first and second chambers and controlling flow passages therethrough, wherein
the outflow tube of the first chamber is connected to the blood dialyzing filter and the inflow tube of the second chamber is connected to the blood dialyzing filter.

2. The blood dialyzing apparatus of claim 1, wherein the outflow tubes of the first and second chambers are connected to the respective chambers at an upper portion of the chambers along a vertical direction.

3. The blood dialyzing apparatus of claim 2, wherein the first chamber is compressed while the second chamber is expanded.

4. The blood dialyzing apparatus of claim 3, wherein the flow control unit further includes a fluid pump transferring dialysate to the first chamber or a second fluid pump.

5. The blood dialyzing apparatus of claim 3, wherein the flow control unit further includes:
a first fluid container having an internal space to accommodate the dialysate,
a second fluid container having an internal space to store the dialysate, and
a load cell measuring a weight of the first fluid container and the second fluid container, wherein
the first fluid container is placed upstream of the first chamber, allowing the dialysate of the first fluid container to be supplied to the first chamber, and the second fluid container is placed downstream of the second chamber, thereby allowing the dialysate of the second chamber to flow into the second fluid container,
the first and second fluid containers are each connected with an inlet tube and an outlet tube, wherein a fluid is supplied to the container through the inlet tube and the fluid of the chamber is discharged through the outlet tube;
the first and second fluid containers are each connected with a vent tube allowing air or dialysate to flow therethrough,
flow control valves are installed in the inlet and outlet tubes connected to the first and second fluid containers, and
a fluid pump is provided to transfer dialysate to the first fluid container or discharge dialysate from the second fluid container.

6. The blood dialyzing apparatus of claim 5, wherein the load cell further comprises a first load cell measuring a weight of the first fluid container and a second load cell measuring a weight of the second fluid container, wherein
the first load cell is placed at the bottom of the first fluid container and the second load cell is positioned at the bottom of the second fluid container.

7. The blood dialyzing apparatus of claim 5, wherein the first and second fluid containers are stacked vertically, and the load cell is placed at the bottom of the stacked fluid containers.

8. The blood dialyzing apparatus of claim 2, wherein the flow control unit further includes:
first and second volume chambers each having an internal space, and
a volume chamber pressurizing members disposed inside each of the first and second volume chambers to compress and expand the internal spaces thereof, wherein
the first volume chamber is connected to a tube connecting the first chamber and the blood dialyzing filter, and the second volume chamber is connected to a tube connecting between the blood dialyzing filter and the second chamber, and
a flow control valve is installed in the tube connected to the second volume chamber.

9. The blood dialyzing apparatus of claim 8, wherein the first and second chambers are compressed and expanded simultaneously, and the volume chambers are compressed and expanded simultaneously, wherein
when the first and second chambers are compressed, the first and second volume chambers are expanded.

10. The blood dialyzing apparatus of claim 9, wherein
the second volume chamber is connected with a second tube through which dialysate of the second volume chamber is discharged when the second volume chamber is compressed, and
a second flow control valve is installed in the second tube connected to the second volume chamber.

11. The blood dialyzing apparatus of claim 10, wherein the flow control unit further includes:
a first fluid container having an internal space to accommodate the dialysate,
a second fluid container having an internal space to store the dialysate, and
a load cell measuring a weight of the first fluid container and the second fluid container, wherein
the first fluid container is placed upstream of the first chamber, allowing the dialysate of the first fluid container to be supplied to the first chamber, and the second fluid container is placed downstream of the second chamber, thereby allowing the dialysate of the second chamber to flow into the second fluid container,
the first and second fluid containers are each connected with an inlet tube and an outlet tube, wherein a fluid is supplied to the container through the inlet tube and the fluid of the chamber is discharged through the outlet tube;
the first and second fluid containers are each connected with a vent tube allowing air or dialysate to flow therethrough,
flow control valves are installed in the inlet and outlet tubes connected to the first and second fluid containers, and
a fluid pump is provided to transfer dialysate to the first fluid container or discharge dialysate from the second fluid container.

12. The blood dialyzing apparatus of claim 11, wherein the load cell further comprises a first load cell measuring a weight of the first fluid container and a second load cell measuring a weight of the second fluid container, wherein
the first load cell is placed at the bottom of the first fluid container and the second load cell is positioned at the bottom of the second fluid container.

13. The blood dialyzing apparatus of claim 11, wherein the first and second fluid containers are stacked vertically, and the load cell is placed at the bottom of the stacked fluid containers.

14. A blood dialyzing apparatus comprising:
a blood dialyzing filter;
a blood flowing unit where blood flows through the blood dialyzing filter, and
a dialysate flowing unit where dialysate is prepared and transferred through the blood dialyzing filter, the dialysate flowing unit including a flow control unit for regulating the dialysate flow through the blood dialyzing filter, wherein
the flow control unit comprising:
a first fluid container having an internal space to accommodate dialysate,
a second fluid container having an internal space to store dialysate, and
a load cell measuring a weight of the first fluid container and the second fluid container, wherein
the first fluid container is placed upstream of the blood dialyzing filter and the second fluid container is placed downstream of the blood dialyzing filter,
the first and second fluid containers are each connected with an inlet tube and an outlet tube, allowing dialysate to flow into and flow out of the container, respectively, and a vent tube allowing air or dialysate to flow therethrough,
the first and second fluid containers are each provided with a level sensor installed in a container housing and detecting a fluid level therein, and
flow control valves are installed in the inlet and outlet tubes connected to each of the first and second fluid containers.

15. The blood dialyzing apparatus of claim 14, wherein the load cell further comprises a first load cell measuring a weight of the first fluid container and a second load cell measuring a weight of the second fluid container, wherein
the first load cell is positioned at the bottom of the first fluid container and the second load cell is positioned at the bottom of the second fluid container.

16. The blood dialyzing apparatus of claim 14, wherein the first and second fluid containers are assembled with each other, and
the load cell is positioned at the bottom of the assembled fluid containers.

17. The blood dialyzing apparatus of claim 16, wherein the first and second fluid containers are further provided with a protruding body placed inside the container at a fluid level of the level sensor.

18. The blood dialyzing apparatus of claim 16, wherein each of the fluid containers includes two or more level sensors installed inside a container housing, wherein at least one level sensor is placed at a lower portion inside the container housing along a vertical direction and another level sensor is placed at an upper portion inside the container housing, wherein
the second fluid container include equal to or greater number of level sensors than the first fluid container.

19. The blood dialyzing apparatus of claim 16, wherein the flow control unit further includes a weight with a known mass to calibrate the load cell.
